(19) United States
(12) Patent Application Publication
(10) Pub. No.: US 2006/0062725 A1
(43) Pub. Date: Mar. 23, 2006

(54) Methods for preparing metal-carborane complexes for radioimaging and radiotherapy

(76) Inventors: John F. Valliant, Ancaster (CA); Oyebola O. Sogbein, North Bay (CA)

Correspondence Address: BERESKIN AND PARR, 40 KING STREET WEST, BOX 401, TORONTO, ON, M5H 3Y2, CA

(21) Appl. No.: 11/199,359

(22) Filed: Aug. 9, 2005

Related U.S. Application Data
(60) Provisional application No. 60/599,556, filed on Aug. 9, 2004.

(51) Int. Cl. A61K 51/00; C07F 13/00; C07F 5/00

(52) U.S. Cl. 424/1.11; 534/11; 534/14

(57) ABSTRACT

The present invention relates to a method of preparing metal-carborane complexes comprising reacting a salt of the formula:
[M(CO)₃(X^{m})₃]^{(1+3m)}, wherein M is selected from a radioisotope of rhenium (Re), technetium (Tc) and any other radioisotope binding in the same fashion, X is the same of different and is, independently, any suitable ligand and m is the formal charge for ligand X, with a nido-carborane or a closo-carborane in the presence of a hard base. In an embodiment of the invention, the hard base is a source of fluoride. Such a method is useful for preparing complexes suitable for radioimaging and radiotherapy.

### CROSS-REFERENCE TO RELATED APPLICATION

The present application claims priority on U.S. provisional application No. 60/599,556 filed on Aug. 9, 2004. This provisional application is incorporated herein by reference in its entirety.

### FIELD OF THE INVENTION

The present invention relates to improved methods for the preparation of radiopharmaceuticals, in particular carborane complexes of technetium and rhenium.

### BACKGROUND OF THE INVENTION

Cyclopentadienide (Cp⁻) complexes of ^{99m}Tc(Eγ=141 keV, t_{1/2}=6.02 h), the most widely used radionuclide in diagnostic medicine (Jurisson, S. S.; Lydon, J. D. Chem. Rev. 1999, 99, 2205) are attractive synthons for the development of organometallic radiopharmaceuticals because of the metal complexes' small size and stability. A number of synthetic approaches to CpTc(CO)₃ and related derivatives have been developed [(a) Wenzel, M. J. Labelled Compd. Radiopharm. 1992, 31, 641. (b) Spradau, T. W.; Katzenellenbogen, J. A. Organometallics 1998, 17, 2009. (c) Cesati, R. R., III; Katzenellenbogen, J. A. J. Am. Chem. Soc. 2001, 123, 4093. (d) Minutolo, F.; Katzenellenbogen, J. A. J. Am. Chem. Soc. 1998, 120, 4514. (e) Minutolo, F.; Katzenellenbogen, J. A. Angew. Chem., Int. Ed. 1999, 38, 1617. (f) Spradau, T. W.; Edwards, W. B.; Anderson, C. J.; Welch, M. J.; Katzenellenbogen, J. A. Nucl. Med. Biol. 1999, 26, 1. (g) Cesati, R. R., III; Tamagnan, G.; Baldwin, R. M.; Zoghbi, S. S.; Innis, R. B.; Kula, N. S.; Baldessarini, R. J.; Katzenellenbogen, J. A. Bioconjugate Chem. 2002, 13, 29. (h) Thei, M.; Kothari, K.; Pillai, M. R. A.; Hassan, A.; Sarma, H. D.; Chaudhari, P. R.; Unnikrishnan, T. P.; Korde, A.; Azzouz, Z. J. Labelled Compd. Radiopharm. 2001, 44, 603.]; however, because cyclopentadiene (Cp) does not react efficiently with metals in aqueous media and because its conjugate base oligomerizes in water, these methods typically require the use of organic solvents, harsh reagents and reaction conditions, and/or multiple synthetic steps, which limits their applicability for routine clinical use. More recently, Alberto and colleagues showed that introduction of an electron withdrawing carbonyl substituent on the Cp ring stabilizes the conjugate base to the extent that it facilitates the direct synthesis of RC(O)CpM(CO)₃ (M=Re, ⁹⁹Tc, ^{99m}Tc) complexes in water in reasonable yields (Wald, J.; Alberto, R.; Ortner, K.; Candreia, L. Angew. Chem., Int. Ed. 2001, 40, 3062). Half-sandwich complexes of technetium linked to a serotonergic ligand were prepared using this approach (Bernard, J.; Ortner, K.; Spingler, B.; Pietzsch, H.-J.; Alberto, R. Inorg. Chem. 2003, 42, 1014).

As an initial step toward establishing a new class of Tc organometallic radiopharmaceuticals, the present inventors developed a method for preparing ⁹⁹Tc(I) (E_{βmax}=294 keV, t_{1/ 2}=2.13×10⁵ yr) and Re(I)-carborane complexes in water [(a) Valliant, J. F.; Morel, P.; Schaffer, P.; Kaldis, J. H. Inorg. Chem. 2002, 41, 628; (b) Valliant, J. F.; Morel, P.; Schaffer, P.; Sogbein, O.O. U.S. patent application Publication No. 2003-0668271, published Apr. 10, 2002)]. The carborane ligands, which are prepared by deboronation of dicarba-closododecaboranes followed by deprotonation of the resulting nido-carboranes, are isolobal to Cp⁻, but, unlike the quintessential organometallic ligand, they are highly effective at forming metal complexes in water (Grimes, R. N. Coord. Chem. Rev. 2000, 200-202, 773). One further advantage to using carboranes over more traditional ligands is that they can be readily functionalized with a wide range of different groups at select vertices regioselectively (Valliant, J. F.; Guenther, K. J.; King, A. S.; Morel, P.; Schaffer, P.; Sogbein, O. O.; Stephenson, A. Coord. Chem. Rev. 2002, 232, 173). This affords a tremendous amount of flexibility when designing novel radiopharmaceuticals [(a) Hawthorne, M. F.; Maderna, A. Chem. Rev. 1999, 99, 3421. (b) Wilbur, D. S.; Chyan, M.-K.; Hamlin, D. K.; Kegley, B. B.; Risler, R.; Pathare, P. M.; Quinn, J.; Vessella, R. L.; Foulon, C.; Zalutsky, M.; Wedge, T. J.; Hawthorne, M. F. Bioconjugate Chem. 2004, 15, 203. (c) Eriksson, L.; Tolmachev, V.; Sjo\ddot{}berg, S. J. Labelled Compd. Radiopharm. 2003, 46,623.]

Metallocarboranes are typically prepared in the presence of strong bases in order to remove the bridging proton on the nido-carborane ligand [(a) Hawthorne, M. F.; Young, D. C.; Andrews, T. D.; Howe, D. V.; Pilling, R. L.; Pitts, A. D.; Reintjes, M.; Warren, L. F., Jr.; Wegner, P. A. J. Am. Chem. Soc. 1968, 90, 879. (b) Hawthorne, M. F.; Varadarajan, A.; Knobler, C. B.; Chakrabarti, S.; Paxton, R. J.; Beatty, B. G.; Curtis, F. L. J. Am. Chem. Soc. 1990, 112, 5365. (c) Valliant, J. F.; Morel, P.; Schaffer, P.; Sogbein, O.O. U.S. patent application Publication No. 2003-0668271, published Ap. 10, 2002)]. The ⁹⁹Tc-carborane complexes reported previously were prepared in water in the presence of KOH or Na₂CO₃ and [⁹⁹Tc(CO)₃Br₃]²⁻. When the analogous reactions were carried out at the tracer level with [^{99m}Tc(CO)₃(OH₂)₃]⁺ [(a) Alberto, R.; Schibli, R.; Egli, A.; Schubiger, P. A. J. Am. Chem. Soc. 1998, 120, 7987. (b) Alberto, R.; Ortner, K.; Wheatly, N.; Schibli, R.; Schubiger, A. P. J. Am. Chem. Soc. 2001, 123, 3135.] the yields of the desired products were low (less than 10%) with the major reaction product being ^{99m}TcO₄⁻. Changing the amount of ligand, reaction time, pH, or temperature did not produce yields comparable to those observed for reactions performed at the macroscopic scale with ⁹⁹Tc or cold Re.

Reports of the direct formation of a metallocarborane from the corresponding closo-isomer are rare (Hawthorne, M. F. J. Organomet. Chem. 1975, 100, 97.) and there currently exists no method to carry out such a reaction in water.

There remains a need for an efficient synthesis of metallocarboranes, in particular of Tc and Re, in aqueous solutions at the tracer levels that provides good yields of the desired products.

### SUMMARY OF THE INVENTION

A new method for the preparation of metallocarboranes in water under mild reaction conditions has been developed. Three nido-carborane ligands were reacted with [Re(CO)₃Br₃]²⁻ in the presence of aqueous potassium fluoride, a hard base, to give the corresponding η⁵-Re(CO)₃-carborane complexes. The use of KF as a base afforded the desired Re-metallocarboranes in good yields while avoiding the formation of Re clusters, which are byproducts commonly observed when reactions are carried out in the presence of strong, soft aqueous bases. The reaction was also performed at the tracer level producing the first ^{99m}Tc-carborane complex, which was isolated in 80% radiochemical yield following a simple Sep-Pak™ purification process. The resulting organometallic complex was stable to cysteine and histidine challenges for more than 24 hours. It was also found that it is possible to prepare the metallocarboranes directly from the closo-carboranes, in a single step, using substantially the same conditions as for the nido-carboranes.

Accordingly, the present invention relates to a method of preparing metal-carborane complexes comprising reacting a salt of the formula:
[M(CO)₃(X^{m})₃]^{(1+3m)},
wherein M is selected from a radioisotope of rhenium (Re), technetium (Tc) and any other radioisotope binding in the same fashion:, X is the same or different and is, independently, any suitable ligand and m is the formal charge for ligand X, with a nido-carborane or a closo-carborane in the presence of a hard base. In an embodiment of the invention, the base is a source of fluoride (F⁻), for example potassium fluoride. In a further embodiment of the invention, the metal, M, is selected from radioisotopes of Tc and Re.

The present invention also includes a kit for use in the preparation of the salts of formula [M(CO)₃(X^{m})₃]^{(1+3m)}, wherein M is a radioisotope of Tc or Re and X is H₂O, comprising potassium boranocarbonate (K₂H₃BCO₂), Na₂B₄O₇.10H₂O, Na₂CO₃ and a hard base, for example a source of F⁻.

The present invention also relates to the use of a fluoride anion for stabilizing a salt of formula:
[M(CO)₃(X^{m})₃[^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X.

The present invention also relates to a method for stabilizing a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, comprising combining the salt with a fluoride anion.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows γ-HPLC radiochromatograms of (A) the crude reaction mixture after 3 h at 85° C.; (B) Sep-Pak purified 2d; and (C) UV-HPLC chromatogram of the Re standard 2b.

FIG. 2 shows γ-HPLC radiochromatograms of 2d after 24 h of L-cysteine (A) and L-histidine (B) challenges.

FIG. 3 showns a X-Ray structure of 2c showing 30% thermal probability ellipsoids.

FIG. 4 shows γ-HPLC radiochromatograms showing the conversion of [^{99m}TcO₄]⁻ (t_{R}=11 min) to [^{99m}Tc(CO)₃(OH₂)₃]⁺ (t_{R}=4.9 min) with increasing fluoride ion concentrations (method C elution conditions): 260 mM (front), 404 mM (second from front), 500 nM (second from back), 1215 nM (back).

FIG. 5 shows γ-HPLC radiochromatograms showing the conversion of [^{99m}TcO₄]⁻ (t_{R}=11 min) to [^{99m}Tc(CO)₃(OH₂)₃]⁺ (4.9 min) at different pH values (method C elution conditions):10-10.5 (front), 9-9.5 (second from front), 8-8.5 (second from back), 7-7.5 (back).

FIG. 6 shows [Front] γ-HPLC radiochromatogram of the crude reaction mixture after 3 hours at 85° C.; [Middle] γ-HPLC radiochromatogram of Sep-Pak® purified 23b; [Back] UV-HPLC chromatogram of the Re-standard (method A elution conditions).

FIG. 7 shows formation of 23b as a function of pH and ligand concentration for the reaction of 1b with [^{99m}Tc(CO)₃(OH₂)₃]⁺ after 3 hours at ligand concentrations of: (▴) 10⁻⁴ M, (•) 10⁻³ M, and (▪) 10⁻² M.

FIG. 8 shows γ-HPLC radiochromatograms showing the formation of 23b at: [A] 10 mM NaF, [B] 50 mM NaF, [C] 100 mM NaF, [D] 500 mM NaF, and [E] 1000 mM NaF (pH≧12; [L]=10⁻³ M) (method A elution conditions).

FIG. 9 shows [A] γ-HPLC radiochromatogram of the crude reaction mixture after 90 minutes; [B] γ-HPLC radiochromatogram of Sep-Pak® purified 5; [C] UV-chromatogram of the Re-standard (method A elution conditions).

### DETAILED DESCRIPTION OF THE INVENTION

The present inventors believed that the unacceptably low yields of ⁹⁹Tc-carborane complexes prepared in water in the presence of KOH or Na₂CO₃ and [⁹⁹Tc(CO)₃Br₃]²⁻ obtained at the tracer level might have been due to the highly basic reaction conditions which can cause decomposition of [^{99m}mTc(CO)₃(OH₂)₃]⁺ prior to complex formation. In light of these results, a search for a base that would remove the bridging hydrogen on the carborane, which is needed for efficient complexation, but that would not cause premature decomposition of the Tc starting material was initiated.

Fluoride ion (pKb≈10.8) is one of the few bases that does not react with the M(CO)₃⁺ core (M=Re, Tc), making it a plausible candidate for preparing metallocarboranes in aqueous solutions (Salignac, B.; Grundler, P. V.; Cayemittes, S.; Frey, U.; Scopelliti, R.; Merbach, A. E.; Hedinger, R.; Hegetschweiler, K.; Alberto, R.; Prinz, U.; Raabe, G.; Ko\ddot{}lle, U.; Hall, S. Inorg. Chem. 2003, 42, 3516.). Bases that are commonly used to prepare metallocarboranes (n-BuLi, NaH, TIOEt, and NaOH) [(a) Hawthorne, M. F.; Andrews, T. D. J. Am. Chem. Soc. 1965, 87, 2496. (b) Ellis, D. D.; Jelliss, P. A.; Stone, F. G. A. Organometallics 1999, 18, 4982.] are much stronger and softer than fluoride; consequently, the question remained as to whether or not an aqueous fluoride solution would be able to generate sufficient amounts of the deprotonated nido-carborane, otherwise referred to as the nido-dicarbollide dianion, to afford good yields of the desired products. To probe the feasibility of using fluoride as a base, the nido-carborane ligands 1a and 1b were reacted with [NEt₄]₂[Re(CO)₃Br₃] in the presence of 100 mM KF at 85° C. (Scheme 1).

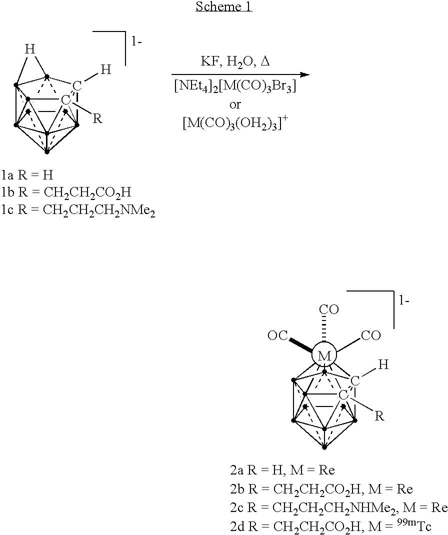

It has been previously demonstrated that these particular carborane ligands, under strongly basic conditions, react with the Re(CO)₃⁺ core to give the corresponding η⁵-metal complexes in good yield. After heating, the desired products, 2a and 2b, were obtained in 34% and 50% isolated yields, respectively. In the case of the reaction involving lb, analysis of the crude reaction mixture by HPLC indicated a yield of greater than 80%; however, difficulties were encountered in separating the different salts of 2b, which in turn reduced the overall isolated yield. The yield of the unsubstituted carborane was also compromised by the desire to isolate a single salt and by the low solubility of the ligand in aqueous KF. Nonetheless, the yields of the Re-metallocarboranes were still an improvement over those values reported for the direct synthesis of CpRe(CO)₃ type complexes in water. One major advantage to using fluoride as a base is that it does not promote the formation of polynuclear hydrolysis products. [Re(CO)₃(OH₂)₃]⁺, which is formed when [Re(CO)₃Br₃]²⁺ is dissolved in dilute aqueous solutions, is stable below pH 6. In the presence of hydroxide ion, deprotonation of the metal bound water molecules occurs, which in turn leads to the formation of Re clusters like [Re₃(CO)₉(μ₂-OH)₃(μ₃-OH)]⁻ [(a) Alberto, R.; Egli, A.; Abram, U.; Hegetschweiler, K.; Gramlich, V.; Schubiger, P. A. J. Chem. Soc., Dalton Trans. 1994, 2815. (b) Egli, A.; Hegetschweiler, K.; Alberto, R.; Abram, U.; Schibli, R.; Hedinger, R.; Gramlich, V.; Kissner, R.; Schubiger, P. A. Organometallics 1997, 16, 1833.] Until now, the formation of these metal clusters complicated the use of ligands, like carboranes, which require the removal of weakly acidic protons prior to complexation. For example, when compound 1c, which is a carborane analogue of the monoamine oxidase-B (MAO-B) inhibitor N,N-dimethyl-3-phenylpropylamine (Ding, C. Z.; Lu, Z.; Nishimura, K.; Silverman, R. B. J. Med. Chem. 1993, 36, 1711.), was reacted with [NEt₄]₂[Re(CO)₃Br₃] in the presence of aqueous NaOH (pH 12), the major products were a series of rhenium clusters. When 1c and [NEt₄]₂[Re(CO)₃Br₃] were combined in the presence of 100 mM KF and the mixture was heated for 13 h, analysis of the crude reaction mixture by electrospray mass spectrometry showed only the desired product and no evidence of any cluster formation. The pH of the reaction mixture was subsequently adjusted to 5 by the dropwise addition of 1 M HCl, and the product 2c, as the internal salt, was isolated in good yield (70%). Compound 2c, which is a novel metallocarborane derivative, is stable in the solid state and in solution. The IR shows the characteristic B—H stretch at 2526 cm⁻¹, which is not significantly shifted from that of the ligand. The CO peaks appear at 2006 and 1899 cm⁻¹ with relative intensities that are consistent with the local symmetry of the metal complex. The electrospray mass spectrum showed the predicted molecular ion having the appropriate isotope distribution while ¹H, ¹¹B, and ¹³C NMR spectra were consistent with the structure of the target compound.

Accordingly, the present invention relates to a method of preparing metal-carborane complexes comprising reacting a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, with a nido-carborane in the presence of a hard base.

In embodiments of the present invention, the nidocarborane used can be an ortho, para or meta isomer. An example using a meta isomer is given in Scheme 2.

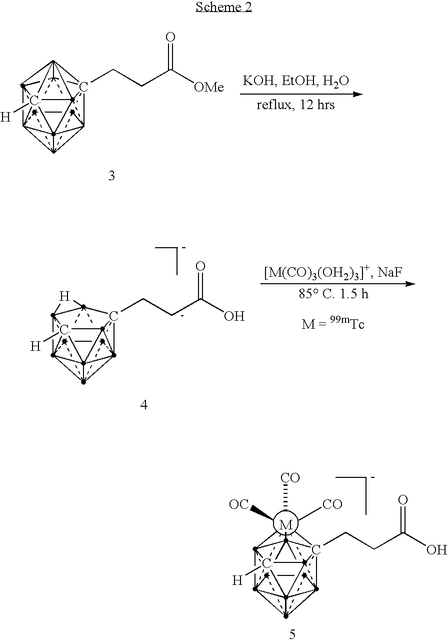

It was also found that it is possible to obtain the desired metal-carboranes as previously defined by using the closo-carboranes directly as an alternative to the use of the isolated nido-carboranes. In fact, it has been found that it is possible to prepare, one pot, the desired metal-carboranes directly form the closo-boranes.

Accordingly, the present invention also relates to a method of preparing metal-carborane complexes comprising reacting a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, with a closo-carborane in the presence of a hard base.

This approach offers a number of advantages. In fact, it represents a reduction in the number of steps necessary to prepare the desired compounds and it eliminates one of the counterions present in solution.

As shown in Scheme 4, compound 2b was obtained directly from the c/oso-carborane 6. As comparison, this compound was also prepared according to the process defined in Scheme 3.

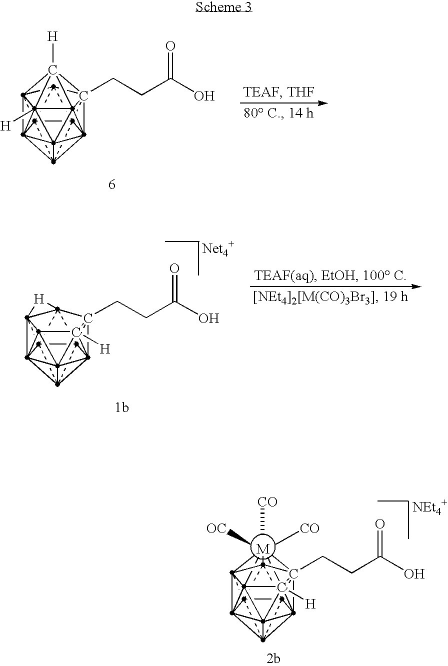

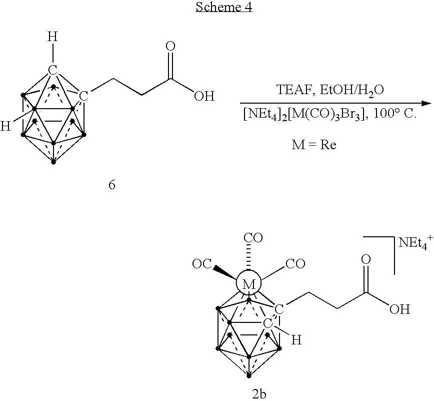

In embodiments of the present invention, the salts of the formula [M(CO)₃(X^{m})₃]^{(1+3m)} include those where M is selected from a radioisotope of rhenium, technetium and any other radioisotope binding in the same fashion. A person skilled in the art would be able to determine which radioisotopes bind in the same fashion as Tc and Re. Examples include radioisotopes of Rh, Cr, Mo, Mn, Os, Ir and Ru. The ligand “X^{m}” may be the same or different (i.e. there may be three different “X” ligands, or one different “X” ligand and two the same or all three 37 X” ligands may be the same) and is, independently, any suitable ligand, including, for example, Cl⁻(m=−1), Br⁻(m=−1), PR₃ (m=0), RCN (m=0), NOₓ^{y} (x=1, 2; y=1, −1) and/or H₂O (m=0). A person skilled in the art would know which ligands are suitable for use in the salts of the formula [M(CO)₃(X^{m})₃]^{(1+3m)}, based on those known in the art. A suitable ligand will be compatible with the reaction conditions used in the method of the invention. It will be appreciated that one or more of the “CO” ligands many be substituted with any ligand that is isoelectronic and isolobal therewith. Examples of such ligands include NO⁺, PR₃, RCN and RNC, wherein R is an alkyl (including cycloalkyl) or aryl group, for example methyl, ethyl, n-butyl, t-butyl and phenyl or R is any biomolecule. The present invention extends to cover the use of salts of the formula [M(CO)₃(X^{m})₃]^{(1+3m)} in which one or more of the CO ligands has been substituted with a ligand that is isoelectronic and isolobal therewith. An example of such a complex wherein one CO has been replaced with NO⁺ is found in Rattat et al. Cancer Biotherapy & Radiopharmaceuticals, 2001, 16(4), 339-343. A person skilled in the art would also understand that the salts of the formula [M(CO)₃(X^{m})₃]^{(1+3m)} may require one or more counterions to balance the charge on the complex. Any such counterion compatible with the reaction conditions may be used in the method of the invention. In an embodiment of the invention, M is selected from radioisotopes of Re and Tc and all three X ligands are H₂O or Br⁻.

The salts of the formula [M(CO)₃(X^{m})₃]^{(1+3m)} may be prepared using methods known in the art. For example, salts of the formula [M(CO)₃Br₃]²⁻ (M=Re, ⁹⁹Tc) may be prepared as described in Alberto et al. (Alberto, R.; Schibli, R.; Egli, A.; Schubiger, P. A.; Herrmann, W. A.; Artus, G.; Abram, U.; Kaden, T. A. J. Organomet. Chem. 1995, 493, 119-127.) In general these compounds may be prepared, for example, using low temperature reductions of [NBu₄][MO₄] in the presence of CO. The reducing agent may be any suitable reagent, such as a boron hydride, including BH₃₋ THF and NaBH₄.

The nido-dicarbolide dianion may be derived from the possible isomers of carborane, for example, ortho-, para- and meta-carborane. The nido-dicarbollide dianions may also be used in optically pure form or as a racemic mixture. The invention includes the use of all isomers and mixtures thereof in any proportion.

In order to be useful as a radiopharmaceutical ligand, it is desirable that a means to conjugate the carborane-M(CO)₃ unit to targeting biomolecules be available. To this end one or more linker moieties may be incorporated into the nido-carborane. Accordingly, the terms “nido-carborane” and “closo-carborane” include those derived from unsubstituted and substituted carboranes, in particular carboranes in which a linker group has been attached to one or more of the carbon and/or boron atoms. The one or more linkers may be the same or different. In embodiments of the present invention, the one or more linker groups are attached to the carbon atoms in the carborane. In further embodiments, one linker group is attached to one of the carbon atoms in the carborane. The terms “nido-carborane” and “closo-carborane” further include those derived from carboranes with one or more linker groups with a biological targeting molecule attached thereto.

As used herein, the term “linker group” means any functional grouping that allows the metal-carborane complex to be conjugated to a biological target ligand. Generally, the linker group will have a reactive functional group at the end opposed to the metal-carborane complex, to allow reaction with (and therefore conjugation to) a reactive functional grouping on the biological target ligand. The one or more linker groups may be the same or different. The specific linker groups used herein comprise a carboxylic acid which is capable of reacting with, for example, free amino, hydroxy or thiol groups on a biological targeting ligand and a hydrazino group, which is capable of reacting with, for example, an aldehyde or other electrophilic group on a biological targeting ligand.

Examples of biological targeting ligands include, but are not limited to, small molecules having specificity for a specific receptor, immunoproteins, oligopeptides, sugars, cocaine analogues, oligopeptides and polypeptides such as epidermal growth factor. The applications of radiolabelled boron clusters to the diagnosis and treatment of cancer has been recently reviewed (Hawthorne, M. F.; Varadarajan, A.; Knobler, C. B.; Chakrabarti, S. J. Am Chem. Soc. 1990, 112, 5365.).

The terms “nido-carborane” and “closo-carborane” further include metal carborane complexes that have been incorporated within the structure of a biological targeting ligand. When the metal-carborane complex is incorporated within the structure of a biological targeting ligand, the ligand is preferably a compound having a functional group that is structurally and electronically similar to the carborane moiety. Examples of such functional groups include phenyl and adamantyl groups. An example of such a ligand is the antiestrogen, tamoxifen. The preparation of carborane analogs of tamoxifen is described in inventor Valliant's publications: Valliant et al. J. Org. Chem. 2002, 67, 383-387, and Valliant, et al., Coord. Chem. Rev. 2002, 232, 173-230. and references cited therein, the contents of which are incorporated herein by reference.

In an embodiment of the present invention, the nido-carborane is selected from compounds 1a, 1b and 1c. The present invention also includes the novel nido-carborane 1c and its corresponding metal carborane complex 2c and 2d. It also includes the novel nido-carborane 9 and 20 and their corresponding metal carborane complex 10 and 21.

In another embodiment of the present invention, the closo-carborane is selected from compounds 3, 6, 8, 11, 12, 15, 17 and 19.

The nido-carboranes can be prepared using procedures known in the art. For example, from the corresponding ortho-, para- and meta-carboranes by a two step process involving deboronation, to provide the dicarba-nido-undecaborate, (for example, [nido-(C₂B₉H₁₂)]⁻), followed by deprotonation using a hard base. The deboronation reaction may be effected using a base under a variety of conditions [(a) Barth, R. F.; Adams, D. M.; Soloway, A. H.; Mechetner, E. B.;Alam, F.; Anisuzzaman, A. K. M. Anal. Chem. 1991, 63, 890. (b) Imahori, Y.; Ueda, S.; Ohmori, Y.; Sakae, K.; Kusuki, T.; Kobayashi, T.; Takagaki, M.; Ono, K.; Ido, T.;Fujii, T. Jpn Clin. Cancer Res. 1998, 4(8), 1833.]. For example, ortho-carborane, para-carborane or meta-carborane may be heated to reflux with potassium hydroxide in an alcoholic solvent, such as ethanol. Other bases that can be used include secondary amines, such as pyrolidine, and fluoride. The dicarba-nido-undecaborate product may be isolated as a salt, for example an ammonium salt such as trimethylammonium, or a phosphonium salt such as methyl triphenylphosphonium, using standard procedures.

The base used for the deprotonation of the dicarba-nido-undecaborate, is suitably a hard base. “Hard” and “soft” are well known terms to describe acids and bases, known as the Hard Soft Acid Base (HSAB) Theory. A person skilled in the art would readily recognize bases that are classified as hard and soft. Hard bases such as fluoride do not react with the soft acid technetium (or rhenium) centre. Hard bases such as fluoride will form hydrogen bonds with the metal bound water but will not deprotonate them because they are not strong bases and therefore will not promote premature degradation of the M(CO)₃⁺ core. Suitable hard bases include, but are not limited to, O²⁻, Cl⁻, F⁻, CH₃COO⁻, NO₃⁻, ClO₄⁻, SO₄²⁻, NH₃ and RNH₂, wherein R is any suitable alkyl (including cycloalkyl) or aryl group, for example methyl, ethyl, butyl, t-butyl, phenyl and the like. In a further embodiment of the present invention, the base is a source of fluoride (F⁻), for example, Z⁺F⁻, wherein Z⁺ is any suitable cation, for example K⁺.

The term “about” as used herein means within experimental error.

Functionalization of the carborane may be performed before or after the formation of the metal complex using methods known in the art (see for example, Hawthorne, M. F.; Maderna, A. Chem. Rev. 1999, 99, 3421-3434 or Hawthorne, M. F.; Varadarajan, A.; Knobler, C. B.; Chakrabarti, S. J. Am Chem. Soc. 1990, 112, 5365.). For example, ortho-carboranes are readily synthesized from the reaction of an appropriately substituted acetylene with various nitrile and sulfide adducts of decaborane (B₁₀H₁₄) (Grimes, R. N. Carboranes, Academic Press, N.Y., 1970; Bregadze, V. I. Chem. Rev. 1992, 92, 209.). The linker group may be incorporated into the carborane by judicious choice of the starting acetylene compound. Hydrophilic groups on the acetylenic compounds should be protected in order that the synthetic sequence will produce the desired orthocarborane. Also, the linker group may be modified using standard procedures at any stage during the preparation of the metal-carborane complex, including modification of the complex itself. The preparation of compounds 1a and 1b has been previously described (Valliant, J. F.; Morel, P.; Schaffer, P.; Sogbein, O. O. U.S. patent application Publication No. 2003-0668271, published Apr. 10, 2002).

The preparation of the metal-carborane complexes may be effected by reacting a nido-carborane with a salt of [M(CO)₃(X^{m})₃]^{(1+3m)} in aqueous solutions. It is an embodiment of the present invention that the reaction is carried out in an aqueous solution of the hard base, for example aqueous KF, suitably 100 mM aqueous KF. The reaction mixture may be warmed and allowed to proceed for a time period of about 30 minutes to about 48 hours, suitably about 1 hour to about 24 hours. The extent of the reaction can be monitored by thin layer chromatography (TLC), therefore a person skilled in the art would be able to determine when the reaction was complete and adjust the reaction time and temperature accordingly. In an embodiment of the invention, the method involves generating the nido-dicarbollide dianion from the corresponding dicarba-nido-undecaborate by treatment with the hard base, for example potassium fluoruide (KF), in aqueous solution, for example aqueous KF solution, and this solution is warmed to a temperature of about 60-100° C., suitably about 80-90° C., more suitably about 85° C., and an aqueous solution of a salt of the formula [M(CO)₃(X^{m})₃]^{(1+3m)}, for example an aqueous KF solution, and warmed to a temperature of about 60-100° C., suitably about 80-90° C., more suitably about 85° C., is added to the solution of the nido-dicarbollide dianion and the temperature maintained for the entire reaction time. The reaction may also be carried out in a microwave as is well known in the art of radiochemical preparation. In further embodiments of the present invention, the reactions are performed in an inert atmosphere, for example under argon (Ar) or nitrogen (N₂) gas. In still further embodiments of the invention, the reactions are performed at tracer levels. By “tracer levels” it is meant that the amount of radiolabeled substances is such that it does not have an effect on the system under study. Typical tracer levels are, for example, in the range of 10⁻⁶ to 10⁻¹² M. The desired product may be purified by any known means, suitably using high performance liquid chromatography (HPLC). As previously indicated, the preparation of the metal-carborane complexes can alternatively be carried out by using the corresponding closo-carboranes instead of the nido-carboranes.

With the success of the reactions involving rhenium, attempts were made to label the bifunctional ligand 1b with [^{99m}Tc(CO)₃(OH₂)₃]⁺, which was prepared using commercially available carbonyl labeling kits. ^{99m}TcO₄⁻(370-740 MBq; 10-20 mCi) was added to the kit and [^{99m}Tc(CO)₃(OH₂)₃]⁺ prepared according to the commercial protocol. After reacting a ligand/KF mixture with [^{99m}Tc(CO)₃(OH₂)₃]⁺, surprisingly, only a small quantity of the desired product was obtained (5% radiochemical yield). After 6 h of heating, the main reaction constituents were unreacted starting materials and ^{99m}TcO₄⁻.

Commercially available kits contain sodium tartrate to prevent premature decomposition of [^{99m}Tc(CO)₃(OH₂)₃]⁺. Thinking that the chelate was interfering with the fluoride mediated complexation reaction, a kit in which KF was substituted for tartrate was prepared. [^{99m}Tc(CO)₃(OH₂)₃]⁺ was successfully prepared using the “KF kit” formulation (see Example 4) in comparable purity and yield to the product from the commercial kit. It should also be noted that [^{99m}Tc(CO)₃(OH₂)₃]⁺ prepared in the presence of KF was stable for greater than 6 h, which is comparable to the stability of the product from the tartrate formulation.

Accordingly, the present invention also includes kits for use in the preparation of the salts of formula [M(CO)₃(X^{m})₃]^{(1+3m)}, wherein M is a radioisotope of Tc or Re and X is H₂O, comprising potassium boranocarbonate (K₂H₃BCO₂), Na₂B₄O₇.10H₂O, a hard base and a suitable buffering reagent, for example Na₂CO₃. In an embodiment of the invention, the amounts of K₂H₃BCO₂, Na₂B₄O₇.10H₂O, base and buffering reagent present are in the ratio of about 8:1:6:5. In a further embodiment of the invention, the hard base is a source of F⁻.

In a typical preparation, an aqueous solution of MO₄⁻, wherein M is a radioisotope of Tc, for example ^{99m}Tc or Re, is added to the kit via syringe, suitably at elevated temperatures, for example about 60-100° C., more suitably about 65-75° C., under an inert atmosphere over a period of about 10 minutes to about 2 hours, suitably about 30 minutes to 1.5 hours, more suitably about 1 hour, to generate the desired salt of the formula[M(CO)₃(X^{m})₃]^{(1+3m)}, wherein M is a radioisotope of Tc or Re and X is H₂O. The reaction may also be performed in a microwave.

In further embodiments of the invention, the kit ingredients are packaged in a suitable container, for example, a glass vial with a rubber stopper as a top, and the container is sold, optionally with directions for use.

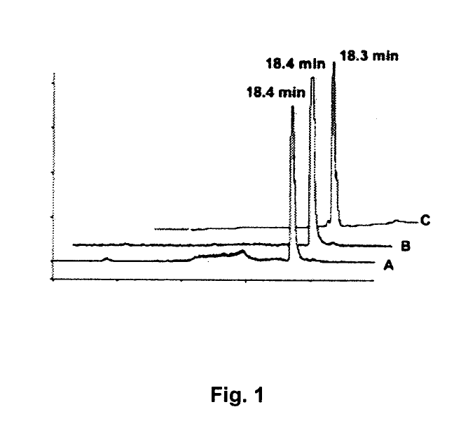

Compound 1b, in the presence of KF, was added to [^{99m}Tc(CO)₃(OH₂)₃]⁺ and the mixture heated for 3 h. The crude γ-HPLC of the reaction mixture showed an appreciable amount of the desired product and only small amounts of residual starting material and ^{99m}TcO₄⁻. The metallocarborane 2d was obtained in 80% isolated yield following purification using a C₁₈ Sep-Pak. The Sep-Pak procedure was able to separate ^{99m}TcO₄⁻ and the excess carborane ligand used during the labeling reaction from the desired product. The γ-HPLC traces of the crude reaction mixture at 3h, the purified product 2d, and the UV trace of the Re standard 2b are shown in FIG. 1. The small difference in retention times between the reference standard and the product is associated with the distance between the UV and γ detectors which are connected in series.

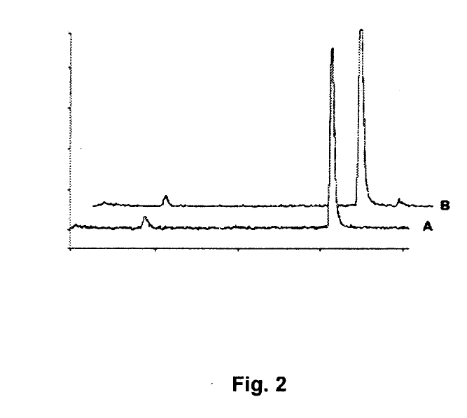

To demonstrate the stability of 2d, the purified product was incubated separately with a 1000-fold excess of cysteine and histidine. Ligand challenge experiments are routinely used in radiopharmaceutical chemistry to determine the likelihood of a compound remaining intact in vivo where there is an abundance of competing thiol and amine ligands. After incubation at 37° C. in phosphate buffered saline (pH=7.2) for 24 h, the radiochromatograms from both experiments indicated that greater than 94% of the product remained unchanged (FIG. 2). These results strongly suggest that the ^{99m}Tc⁻ metallocarborane complexes are sufficiently robust to be used as synthons for preparing radiopharmaceuticals.

The mechanism of the fluoride mediated reaction may not necessarily be a simple acid-base reaction given the weak basicity and acidity of KF and the bridging hydrogen on the carborane, respectively. While not wishing to be limited by theory, two plausible alternatives could be (1) initial η³-coordination of the M(CO)₃⁺ core to the nido-carborane thereby causing a concomitant increase in the acidity of the bridging hydrogen or (2) the presence of KF in solution could generate small quantities of HF, which react with a boron hydride leading to the formation of hydrogen gas and the dicarbollide dianion. The exact details of the mechanism notwithstanding, the reported experiments clearly demonstrate that a hard base such as fluoride, unlike hydroxide ion, does not cause premature decomposition of [M(CO)₃(OH₂)₃]⁺ (M=^{99m}Tc, Re) thereby allowing for the preparation of metallocarboranes at both the macroscopic and tracer levels in water.

As shown in Scheme 3, compound 6 was treated with TEAF in wet THF following the methodology developed by Fox et al. Polyhedron 1997, 16, 2499. Compound 1b was isolated in 64% yield following chromatographic purification which was needed to remove unreacted starting material. Compound 1b was subsequently reacted with [NEt₄]₂[Re(CO)₃Br₃] in a 500 mM solution of TEAF in water/EtOH and the mixture heated to reflux. After approximately 19 hours the product, compound 2b, was isolated in 61% yield. As shown in Scheme 4, compound 6 was combined with a slight excess of [NEt₄]₂[Re(CO)₃Br₃] in a solution of 500 mM TEAF containing a small quantity of absolute ethanol. The heterogeneous suspension was heated at 100° C. and after 30 hours TLC indicated complete consumption of 6. Extraction followed by chromatography lead to isolation of the desired product in 70% yield.

To determine if substituents bearing good donor atoms would impact complexation yields, a pyridine substituted carborane was prepared following a literature procedure ((a) Wang, X.; Jin, G.-X. Organometallics 2004, 23, 6319 (b) Alekseyeva, E. S.; Batsanov, A. S.; Boyd, L. A.; Fox, M. A.; Hibbert, T. G.; Howard, J. A. K.; MacBride, J. A. H.; Mackinnon, A.; Wade, K. Dalton Trans. 2003, 3, 475). Pyridines, which are excellent ligands for Re(l), have been used to construct a number of Re(I) and Tc(I) bifunctional chelators. Pyridine substituted compounds have the added attraction that they can also be used to prepare piperidine derivatives as a means of targeting specific neuroreceptors. The nido-carborane 9 was reacted with [NEt₄]₂[Re(CO)₃Br₃] in the presence of 100 mM KF and the solution heated to reflux for 24 hours. After the addition of HCl to form the internal salt, the product was isolated in excellent yield (85%) by extraction into dichloromethane (Scheme 5).

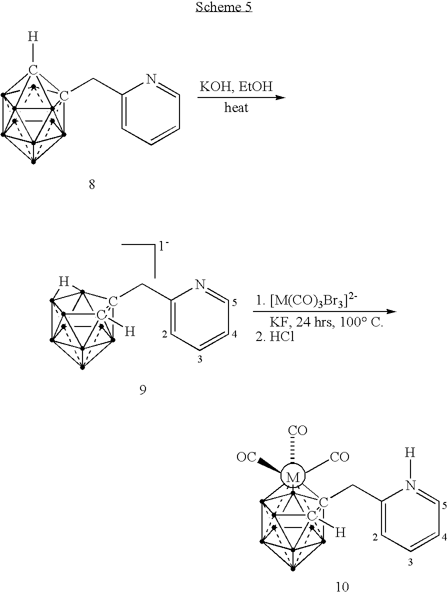

The MS of 10 is consistent with the formation of the η⁵-rhenacarborane complex as opposed to a compound in which Re is coordinated to the pyridine nitrogen. The ¹H NMR showed some minor shifts in the aromatic region of the NMR spectrum compared to that for the starting material, which is expected given the proximity of the pyridine ring to the carborane cage. The ¹H NMR did not show any evidence of the bridging H-atom on the cluster which further supports our hypothesis that metal complex resides on the carborane. The ¹¹B{¹H}NMR of 10 showed six peaks with some overlapping signals that are for the most part shifted to higher frequency compared to that of the starting material.

The higher yield of 10 with respect to the other Re-carborane complexes (vide infra) may be associated with the formation of a kinetic product involving coordination of the pyridyl group to rhenium, which helps prevent the formation of metal clusters. This is analogous to the addition of co-ligands to formulations for preparing Tc(V) complexes as a way of preventing the formation of TcO₂. A further advantage is gained if coordination to pyridine takes place initially in that the intermediate complex would situate the fac-[Re(CO)₃]⁺ core in close proximity to the open C₂B₃ face of the cluster. Alternatively, the pyridine nitrogen may simply facilitate deprotonation of the bridging H-atom during the complexation process. One clear practical advantage is the ability to form the internal salt of 10 which avoided problems associated with the presence of different counter ions.

A carborane bearing a pendent tertiary amine was also prepared and the corresponding [Re(CO)₃]⁺ complex generated. The target Re metallocarborane was prepared from the dimethylamino nido-carborane ligand 1c (Scheme 6), which was synthesized in 58% yield by reacting the iodoalkyl-carborane 11 with an ethanolic solution of dimethylamine overnight at room temperature. Compound 1c and [NEt₄]₂[Re(CO)₃Br₃] were then combined in 500 mM aqueous KF and the resultant heterogeneous mixture heated to 100° C. After 13 hours, the product was isolated as an internal salt by adjusting the pH to 3 by the dropwise addition of HCl. The product was subsequently purified by column chromatography through silica gel to give 2c in 72% yield.

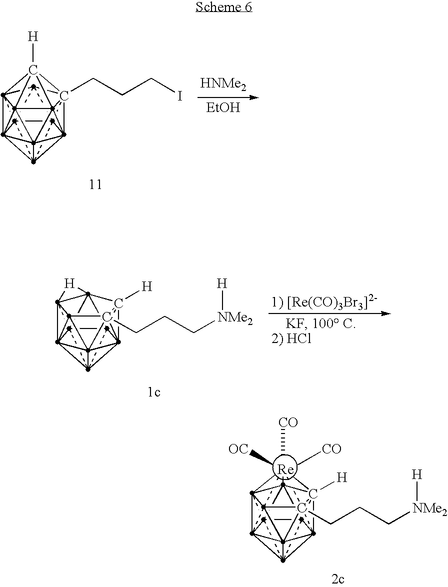

The FTIR of 2c showed the characteristic C≡O stretches at 2005 and 1899 cm⁻¹ while the B—H stretch (2526 cm⁻¹) was not significantly shifted from that of the free ligand. The ¹H NMR revealed that the methylene protons directly adjacent to the cage are diastereotopic and appear as two distinct multiplets at 1.53 and 1.68 ppm. The protons in the methylene group adjacent to the amine in contrast are homotopic. The presence of the protonated amine is evident in that the N-methyl groups, which appear at 3.18 ppm, are split into a doublet from the adjacent N—H group. The amino N—H proton appears as a broad singlet at 5.70 ppm. Its identity was confirmed by adding a small quantity of CD₃OD to the NMR sample which caused the resonance to disappear due to exchange.

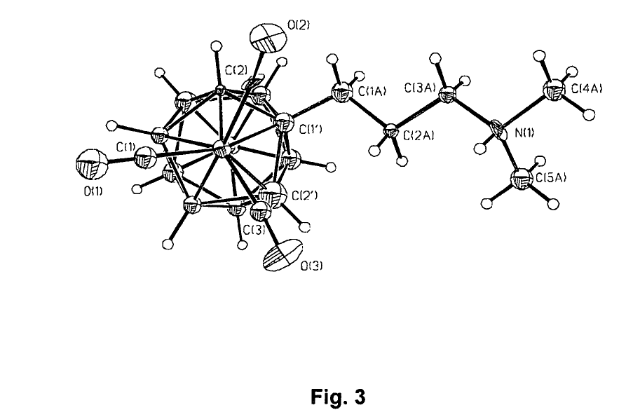

X-ray quality crystals of 2c (see FIG. 3) were obtained using a 1:1 (v/v) solution of dichloromethane and methanol. The structure exhibits the tripodal fac-[Re(CO)₃]⁺ core with one CO ligand nearly eclipsing the carborane C—H bond in the solid state (FIG. 3). The aliphatic chain almost completely bisects the OC—Re—CO bond angle and extends away from the carborane cage. The average Re—B bond distance is 2.322(14) Å which is identical to the Re—C_{cage} distance (2.32(14) Å). Crystallographic data for 2c is summarized in Tables 1 and 2.

TABLE 1
Crystal and structure refinement data for 2c

| Empirical formula | C₁₀H₂₃B₉NO₃Re | |
|---|---|---|
| Formula weight | 488.78 | |
| Temperature | 173(2) K | |
| Wavelength | 0.71073 Å | |
| Crystal system | Orthorhombic | |
| Space group | P2₁2₁2₁ | |
| Unit cell dimensions | a = 8.982(2) Å | α = 90°. |
| | b = 11.563(3) Å | β = 90°. |
| | c = 16.811(4) Å | γ = 90°. |

| | |
|---|---|
| Volume | 1746.1(7) Å³ |
| Z | 4 |
| Density (calculated) | 1.859 Mg/m³ |
| Absorption coefficient | 6.966 mm⁻¹ |
| F(000) | 936 |
| Crystal size | 0.07 × 0.06 × 0.01 mm³ |
| θ range for data collection | 2.14 to 24.00°. |
| Index ranges | −10 ≦ h ≦ 9, −13 ≦ k ≦ 13, |
| | −19 ≦ l ≦ 19 |
| Reflections collected | 11192 |
| Independent reflections | 2709 [R(int) = 0.1363] |
| Completeness to θ = 24.00° | 99.2% |
| Absorption correction | semi-empirical based on equivalents |
| Refinement method | Full-matrix least-squares on F² |
| Data/restraints/parameters | 2709/39/138 |
| Goodness-of-fit on F2 | 1.102 |
| Final R indices [I > 2Σ(I)] | R1 = 0.0684, wR2 = 0.0896 |
| R indices (all data) | R1 = 0.0982, wR2 = 0.0959 |
| Absolute structure parameter | 0.01(3) |
| Extinction coefficient | 0.00076(15) |
| Largest diff, peak and hole | 1.929 and −2.593 e · Å⁻³ |

TABLE 2
Selected bond lengths and angles for compound 2c

| | |
|---|---|
| Re(1)-C(3) | 1.845(17) |
| Re(1)-C(1) | 1.907(16) |
| Re(1)-C(2) | 1.962(17) |
| Re(1)-B(4) | 2.275(17) |
| Re(1)-B(7) | 2.33(2) |
| Re(1)-C(2′) | 2.34(2) |
| Re(1)-C(1′) | 2.348(19) |
| Re(1)-B(11) | 2.361(19) |
| C(1)-O(1) | 1.168(17) |
| C(3)-O(3) | 1.183(18) |
| C(2)-O(2) | 1.115(17) |
| C(2′)-C(1′) | 1.73(2) |
| C(1′)-C(1A) | 1.54(2) |
| C(3A)-N(1) | 1.522(18) |
| C(5A)-N(1) | 1.497(19) |
| N(1)-C(4A) | 1.489(18) |
| C(3)-Re(1)-C(1) | 86.6(7) |
| C(3)-Re(1)-C(2) | 92.7(7) |
| C(1)-Re(1)-C(2) | 88.6(7) |
| C(3)-Re(1)-C(2′) | 82.2(7) |
| C(1)-Re(1)-C(2′) | 140.2(6) |
| C(2)-Re(1)-C(2′) | 129.8(7) |
| C(3)-Re(1)-C(1′) | 111.1(7) |
| C(1)-Re(1)-C(1′) | 161.6(7) |
| C(2)-Re(1)-C(1′) | 95.4(7) |
| C(2′)-Re(1)-C(1′) | 43.4(6) |
| O(1)-C(1)-Re(1) | 174.4(19) |
| O(3)-C(3)-Re(1) | 176.4(15) |
| O(2)-C(2)-Re(1) | 174.2(14) |
| C(1A)-C(1′)-C(2′) | 124.1(15) |
| C(1A)-C(1′)-Re(1) | 109.9(11) |
| C(2′)-C(1′)-Re(1) | 68.1(10) |
| C(1′)-C(1A)-C(2A) | 115.3(12) |
| C(2A)-C(3A)-N(1) | 111.4(11) |
| C(4A)-N(1)-C(5A) | 109.4(13) |
| C(4A)-N(1)-C(3A) | 111.3(10) |
| C(5A)-N(1)-C(3A) | 111.3(12) |

Compound 12, which was prepared following literature methods (Giovenzana, G. B.; Lay, L.; Monti, D.; Palmisano, G.; Panza, L.; Tetrahedron 1999, 55, 14123), was converted to the corresponding nido-caborane as both the potassium and TEA salts. Formation of the potassium salt involved treating compound 12 with KOH in ethanol and heating the mixture to reflux for 12 hours. This method also resulted in the simultaneous deprotection of the acetate esters on C-2, 3, 4 and 6. The nido-carboranyl glucose derivative 13a could then be extracted into methanol, ethanol, acetone or tetrahydrofuran thereby separating the product from residual salts. Further purification was accomplished using silica gel column chromatography and the desired product, 13a, was obtained as a glassy solid in 83% yield (see Scheme 7).

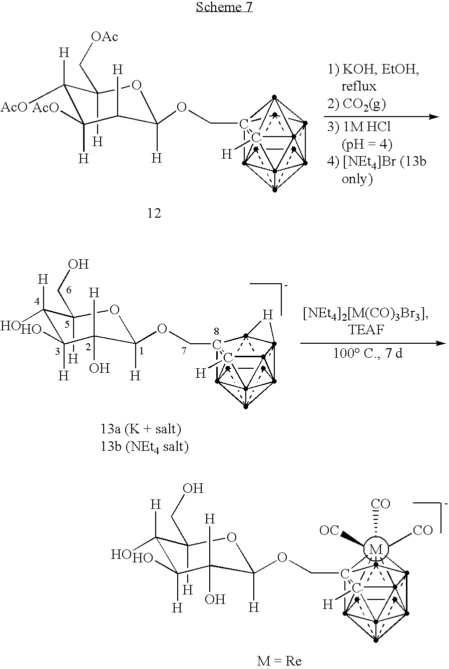

The ¹H NMR of 13a showed the existence of two diastereomers, which arise as a consequence of the fact that during degradation of mono-substituted ortho-carboranes two enantiomers (diastereomers in the case of 13a) are formed. Two distinct signals for the anomeric proton were detected at 4.51 and 4.41 ppm while two pairs of doublets arising from the protons of the C-1 substituent group were also observed. The formation of the nidocarborane was evident in that there was a broad signal at −2.5 ppm which is associated with the hydrogen atom that is bound to the open C₂B₃ face of the nido-carborane cage. The ¹³C NMR spectrum of 13a also indicated a mixture of diastereomers. For instance, there were two signals associated with the anomeric carbon atom at 103.41 and 103.01 ppm and pairs of signals corresponding to the C-3 and C-5 carbon atoms.

Compound 13a and [NE₄]₂[Re(CO)₃Br₃] were combined in 1.0 M aqueous KF and the reaction mixture heated to reflux for 24 hrs. The mass spectrum showed the presence of the ligand mass and the target mass, plus some rhenium cluster species which appeared at m/z=590 and 633, and at 878. Reduction of the concentration of KF to 0.1 M in subsequent reactions appeared to eliminate these undesired products however LC-MS analysis as a function of time showed only very small quantities of the product after 48 hours. After a period of seven days, the peak corresponding to the starting material had diminished almost completely, while the peak corresponding to 14 increased accordingly. The reaction lead to the formation of K⁺ and NEt₄⁺ salts of the desired complex, which were unfortunately inseparable. To simplify purification, the NEt₄⁺ salt (13b) was prepared and the reaction repeated using TEAF as the base. Semi-preparative HPLC was employed to isolate pure 14 in 16% yield. The low yield of the target was somewhat surprising given that the analytical HPLC of the crude reaction mixture indicated a much higher yield than what was actually isolated.

The IR spectrum 14 featured the characteristic O—H stretch at 3425 cm⁻¹, B—H stretches at 2537 cm⁻¹ and C⁰O stretches at 1999 and 1898 cm⁻¹. The electrospray mass spectrum of the purified product showed the target mass with an isotopic distribution characteristic of a ReB₉ cluster. The ¹H and ¹³C NMR spectra, interestingly, appeared to indicate the formation of unequal amounts of the two diastereomers of 14. The anomeric doublets at 4.28 and 4.19 ppm for example appeared with integration ratios of approximately 10:1 in favour of the lower frequency signal. The ¹¹B{¹H} NMR spectrum of 14 showed 7 signals, which appeared at −5.82, −7.65, −8.78, −11.62, −18.35, −19.55, and −20.13 ppm, with the peaks at −8.78 and −11.62 ppm consisting of two overlapping signals. The signals in the ¹¹B{¹H} spectrum for 14 were shifted to higher frequency versus those in 13b.

The long reaction time needed to achieve reasonable yields of 14 could be the result of the formation of an intermediate complex between the rhenium tricarbonyl core and multiple glucose hydroxyl groups. It is reasonable to expect that the product(s) of the rhenium core and the glucose hydroxyl groups would form at a rate that is faster than the formation of the metallocarborane. Separate attempts to isolate a Re-glucose complex however were unsuccessful. With respect to the observed isomer ratio, stereoselectivity in the complexation reaction is improbable. It is more likely that one isomer was enriched during HPLC purification.

One of the attractive features of carboranes is that they can be derivatized at both of the cage carbon atoms selectively as a means of preparing unique targeting agents. Endo et al. have utilized this feature to prepare a series of diphenyl substituted carboranes as estrogen agonists and antagonists ((a) Endo, Y.; Iijima, T.; Yamakoshi, Y.; Fukusawa, H.; Miyaura, C.; Inada, M.; Kubo, A.; Itai, A.; Chem. Biol. 2001, 8, 341 (b) Endo, Y.; Iijima, T.; Yamakoshi, Y.; Kubo, A.; Itai, A. Bioorg. Med. Chem. Lett. 1999, 9, 3313). The metallocarborane complexes of related analogues could serve as a novel class of inorganic antiestrogens ((a) Le Bideau, F.; Salmain, M.; Top, S.; Jaouen, G. Chem. Eur. J. 2001, 7, 2289 (b) Jaouen, G.; Top, S.; Vessières, A.; Alberto, R. J. Organomet. Chem. 2000, 600, 23 (c) Jaouen, G.; Top, S.; Vessieres, A.; Leclercq, G.; McGlinchey, M. J. Current Med. Chem. 2004, 11, 2505) or as radiotracers for imaging estrogen receptor positive tumours (Mull, E. S.; Sattigeri, V. J.; Rodriguez, A. L.; Katzenellenbogen, J. A. Bioorg. Med. Chem. 2002, 10, 1381.) One important consideration for disubstituted derivatives is that the steric hindrance could reduce the yields of the Re complexes. As a consequence, a model compound was prepared and its reactivity towards complexation with the [Re(CO)₃]⁺ core investigated.

Compound 15 was prepared following literature procedures and the synthesis of the target metal complex 16 carried out directly from the c/oso-carborane (Scheme 8). The reaction was performed at reflux using an excess of Re and 500 mM sodium fluoride. After two days, HPLC showed complete consumption of 15 with the target compound being the major product. Compound 16 was obtained via silica gel chromatography as a yellow oil in 51% yield.

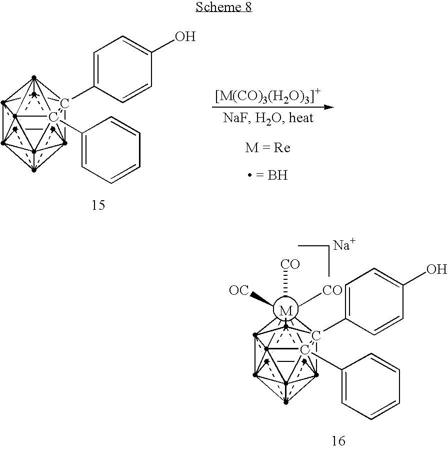

The IR and mass spectra of the Re complex are consistent with the proposed structure of 16. The ¹H NMR spectrum is relatively uncomplicated and shows that the each of the aromatic protons exist in a unique environment. In contrast, the ¹³C NMR spectrum showed multiple environments for most carbon atoms. This is not unexpected as showed that from an orbital overlap perspective, the most favorable conformation of the aryl rings is parallel to the binding face of the carborane ((a) Lewis, Z. G.; Welch, A. J.; J. Organomet. Chem. 1992, 430, C45 (b) Robertson, S.; Ellis, D.; McGrath, T. D; Rosair, G. M.; Welch, A. J. Polyherdron 2003, 22, 1293.). In diaryl carboranes however, interaction between the rings prevents a parallel arrangement. As such, the rings can adopt multiple θ values between 5 and 40° where θ is defined as the angle between the plane made by the ring and the plane defined by the two carbon vertices and the bond to the substituent.

There are three isomeric forms of dicarbaclosododecaborane, which differ in the relative positions of the carbon atoms in the cluster. Sandwich complexes of nido-carboranes derived from the ortho-isomer are widespread while analogous complexes derived from meta-carboranes are comparatively less common. Investigating complexation reactions with the meta isomer, [nido-7,9-C₂B₉H₁₂]⁻ is important because the bonding face of the carborane has a smaller dipole than in the case of [nido-7,8-C₂B₉H₁₂]⁻. This difference could result in more stable metal complexes and higher yields of the desired product. Furthermore, the different relative positions of the carbon atoms in the cluster offers a way to vary the spatial orientations of targeting entities attached to disubstituted carboranes in order to achieve favourable receptor binding interactions.

To determine if the fluoride mediated reaction would work with the meta isomer, the ester 18 (Scheme 9) was prepared and reacted with the [Re(CO)₃]⁺ core. Substituted meta-carboranes are prepared by deprotonating one of the CH vertices of the cluster followed by treatment with an electrophile. In the example presented here, meta-carborane 17 was treated with n-BuLi followed by methyl 3-bromopropionate (Cai, J.; Nemoto, H.; Singaram, B.; Yamamoto, Y. Tetrahedron Lett. 1996, 37, 3383). Compound 18 was purified by silica gel chromatography and recrystallization giving the final product in 46% yield. The complexation reaction was carried out using the c/oso-isomer to allow for direct comparison to the synthesis of compound 2b (Scheme 4). Because saponification of 18 routinely lead to a mixture of the closo and nido acids, the methyl ester itself was used for the complexation reaction. The Re complex was prepared successfully by heating the closo-carborane 18 with [NEt₄][Re(CO)₃Br₃] in a solution of 500 mM TEAF(aq)/absolute EtOH (9:1 v/v) at 100° C. for 22 hours. The meta-rhenacarborane 7 was obtained as a brownish coloured solid in 57% yield after acid hydrolysis of the methyl ester using HCl(aq), which was done to facilitate direct comparison of the spectral data to that of compound 2b.

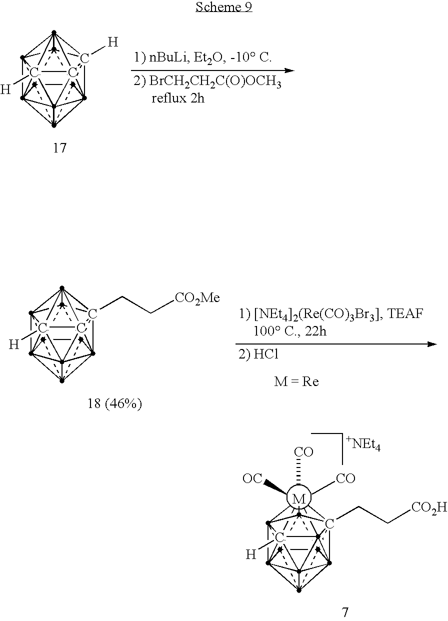

The IR spectrum of compound 7 was nearly identical to that of the ortho-isomer with the primary difference being the position of the two C≡O stretches which appeared at 2032 and 1915 cm⁻¹ in 7 versus 1998 and 1893 cm⁻¹ for 2b. The ¹H NMR spectrum of 7 was also similar to compound 2b where the methylene protons directly adjacent to the carborane cage appeared as two sets of triplets at 2.22 ppm and 2.09 ppm. The carborane C—H was a broad singlet at 1.74 ppm and the protons from the NEt₄⁺ cation were visible as a characteristic quartet (3.48 ppm) and triplet (1.39 ppm). The ¹³C{¹H} NMR spectrum of 7 showed a single resonance for the C≡O carbon atoms at 200.4 ppm which was not significantly shifted from that of the ortho-analogue (199.9 ppm). As expected, the ¹¹B{¹H} NMR spectrum of 7 was significantly different than that for 2b. The difference in the chemical shift particularly for the highest field resonances for 2b in comparison to 7 reflect the differences in the frontier molecular orbitals of the C₂B₃ bonding faces of the two carborane isomers ((a) Batsanov, A. S.; Eva, P. A.; Fox, M. A.; Howard, J. A. K.; Hughes, A. K.; Johnson, A. L.; Martin, A. M.; Wade, K. Dalton Trans. 2000, 3519 (b) Hermánek, S. Inorg. Chim. Acta. 1999, 289, 20). The stability of the resulting complex, at least qualitatively, is comparable to that of the ortho-analogue.

A Cbz-protected hydrazine derivative of meta-carborane 19 (Scheme 10) was prepared using a modification of our published methodology for the Boc analogue. The Cbz protecting group was used in place of Boc groups as we discovered that fluoride in the presence of Re(I) can facilitate deprotection of the t-butylcarbamate (Routier, S.; Saugé, L.; Ayerbe, N.; Coudert, G.; Mérour, J.-Y. Tetrahedron Lett. 2002, 43, 589) even in water, which resulted in the formation of mixtures that included hydrazine-Re complexes. The Cbz-protected hydrazine carborane was subsequently degraded to the corresponding nido-carborane using TEAF in THF in excellent yield. Compound 20 was reacted with [NEt₄]₂[Re(CO)₃Br₃] in varying amounts of TEAF. IR and MS experiments before and after purification indicated the presence of the desired product 21.

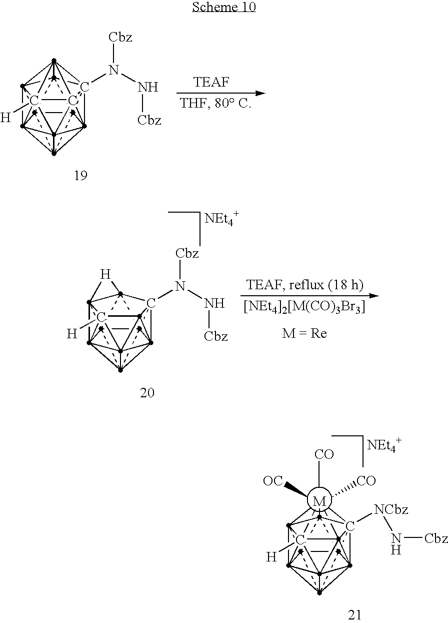

In another experiment, in order to determine if [Tc(CO)₃(OH₂)₃]⁺ could be prepared in the presence of fluoride, and to see if the product would react with a carborane ligand, a series of preliminary reactions were performed using ⁹⁹Tc (E_{βmax}=0.294 MeV, t^{1/2}=2.11×10⁵ yr). This particular isotope of technetium allows reactions to be carried out on a macroscopic scale (i.e. mmol) so that the products can be fully characterized by conventional methods (NMR, mass spectrometry etc.). To this end, [⁹⁹Tc(CO)₃(OH₂)₃]⁺ was synthesized by reacting ⁹⁹TcO₄⁻ with a mixture of NaBH₄, Na₂CO₃ and KF and heating the reaction to 100° C. for 30 minutes (Scheme 11). The final concentration of KF in the reaction vial was 0.1 M. In a separate vial, compound 1b was incubated with 0.1 M KF at 85° C. for one hour and subsequently added via syringe to the vial containing [⁹⁹Tc(CO)₃(OH₂)₃]⁺ and the reaction progress monitored by HPLC. An important observation was that pre-incubating the ligand with fluoride prior to complexation afforded better yields of the desired product for reasons that at present are not obvious.

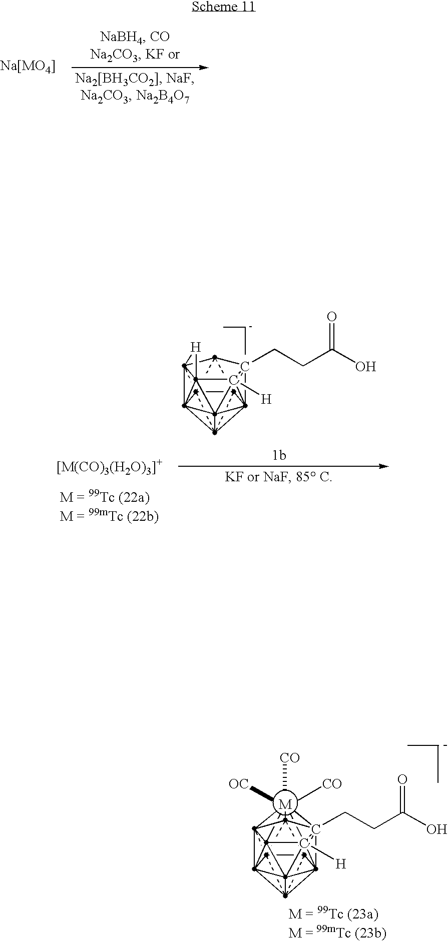

The radiochromatogram (β⁻ detection) of the crude reaction mixture after 14 hours showed a dominant peak at 19.1 minutes, which corresponded to that for the rhenium analogue. Despite the residual [⁹⁹Tc(CO)₃(OH₂)₃]⁺ present in the mixture, the product was readily isolated by HPLC in 25% yield. The negative ion electrospray mass spectrum was consistent with the target mass while the IR spectrum showed the expected features. This includes the carboxylic acid O—H stretch at 3451 cm⁻, the carborane B—H stretch at 2550 cm⁻, and the C≡O stretches at 2017 and 1928 cm⁻.

Based on the success in preparing [⁹⁹Tc(CO)₃(OH₂)₃]⁺ from NaBH₄ in the presence of fluoride, the reaction was repeated at the tracer level using ^{99m}TcO₄⁻. HPLC (y-detection) showed that after 30 minutes the desired product was the main reaction component with unreacted ^{99m}TcO₄⁻ and [^{99m}Tc(CO)₃(OH₂)₃]⁺ being the only impurities. Interestingly, extended incubation of the reaction mixture at elevated temperatures for over three hours resulted in only a small amount of decomposition of the technetium starting material. This observation is in contrast to [^{99m}Tc(CO)₃(OH₂)₃]⁺ prepared in the absence of tartrate and fluoride which decomposed to a much greater extent under the same conditions suggesting that fluoride ion has a unique stabilizing effect on the [Tc(CO)₃]⁺ core.

The commercial kit that is normally used to prepare [^{99m}Tc(CO)₃(OH₂)₃]⁺ consists of Na₂[BH₃.CO₂], Na/K-tartrate, Na₂B₄O₇.10H₂O and Na₂CO₃ ratio of each component had been established for optimal formation of the ^{99m}Tc-trisaquo species. The key ingredient is the boranocarbonate anion (BH₃.CO₂)²⁻, which acts as both the reducing agent (replacing NaBH₄), and the in situ source of CO (Alberto, R.; Ortner, K.; Wheatley, N.; Schibli, R.; Schubiger, A. P. J. Am. Chem. Soc. 2001, 123, 3135). As mentioned previously, when [⁹⁹Tc(CO)₃(OH₂)₃]⁺ prepared from the commercial kit was combined with a nido-carborane, the desired metallocarborane was not detected. Varying the reaction conditions, including the temperature and amount of ligand, did not facilitate the formation of the desired products. Because the boranocarbonate kit is so convenient for the routine preparation of [⁹⁹mTc(CO)₃(OH₂)₃]⁺ and avoids the need to use carbon monoxide, attempts were made to develop the equivalent fluoride-based kits.

Tables 3, 4 and 5 contain the results from the various experiments that were performed to optimize the radiochemical yield of [^{99m}Tc(CO)₃(OH₂)₃]⁺ using fluoride in place of tartrate. Simply replacing tartrate in the commercial kit formulation with an equivalent amount of fluoride did not afford good yields of the desired product. Increasing the quantity of boranocarbonate however, had a dramatic impact on the yield of [^{99m}Tc(CO)₃(OH₂)₃]⁺ (entries 4-6), which improved to 55%. After establishing the need to increase the amount of boranocarbonate, the next step was to adjust the quantity of borate. Borate acts to degrade excess boranocarbonate and prevent unwanted side-reactions with the Tc(l) cation once it has formed. The correct quantity of borate is crucial since an excess would act to degrade boranocarbonate too rapidly, whereas an insufficient quantity might lead to unwanted side-products. The commercial kit utilizes the decahydrate, Na₂B₄O₇.10H₂O, and its molar ratio to Na₂[BH₃.CO₂] approximately 1:6. Using this ratio as a starting point, only poor yields of the [⁹⁹m Tc(CO)₃(OH₂)₃]⁺ cation were obtained. However, subsequent experiments established that higher yields of [^{99m}mTc(CO)₃(OH₂)₃]⁺ could be achieved when using reduced amounts of the anhydrous salt Na₂B₄O₇ instead of the decahydrated form (entries 7-9). In the end, the optimal ratio of anhydrous sodium borate to boranocarbonate was determined to be approximately 1:5, which prompted an investigation of the effect of fluoride ion concentration on the yield of [^{99m}Tc(CO)₃(OH₂)₃]⁺.

TABLE 3
Radiochemical yield of 22b using different formulations (pH ≧ 12).

| | Expt # | | | | | | | | | | | |
|---|---|---|---|---|---|---|---|---|---|---|---|---|
| Reagent | 1 | 2 | 3 | 4 | 5 | 6 | 7 | 8 | 9 | 10 | 11 | 12 |
| K₂[BH₃.CO₂] | 4.0 | 4.0 | 4.0 | 5.5 | 7.5 | 8.5 | 8.5 | 8.5 | 8.5 | 8.5 | 8.5 | 8.5 |
| (mg) | | | | | | | | | | | | |
| Na₂B₄O₇ | 3.5 | 4.5 | 3.0 | 3.0 | 3.0 | 3.0 | 2.5 | 2.2 | 1.9 | 1.9 | 1.9 | 1.9 |
| (mg) | | | | | | | | | | | | |
| NaF (mg) | 2.5 | 2.5 | 2.5 | 2.5 | 2.5 | 2.5 | 2.5 | 2.5 | 2.5 | 3.5 | 5.5 | 7.5 |
| Na₂CO₃ | 4.0 | 4.0 | 4.0 | 4.0 | 4.0 | 4.0 | 4.0 | 4.0 | 4.0 | 4.0 | 4.0 | 4.0 |
| (mg) | | | | | | | | | | | | |
| Yield (%) | 20 | 15 | 25 | 35 | 55 | 55 | 75 | 80 | 80 | 80 | 85 | 85 |

TABLE 4
Radiochemical yield of 22b as a function of fluoride concentration
(pH ≧ 12).

| | Expt. # | | | | |
|---|---|---|---|---|---|
| Reagent | 13 | 14 | 15 | 16 | 17 |
| K₂[BH₃.CO₂] | 8.5 | 8.5 | 8.5 | 8.5 | 8.5 |
| (mg) | | | | | |
| Na₂B₄O₇ | 1.9 | 1.9 | 1.9 | 1.9 | 1.9 |
| (mg) | | | | | |
| NaF (mg) | 8.5 | 9.5 | 10.5 | 11.5 | 25.5 |
| Na₂CO₃ (mg) | 4.0 | 4.0 | 4.0 | 4.0 | 4.0 |
| Yield (%) | 90 | 95 | 95 | 95 | 95 |

TABLE 5
Radiochemical yield of 22b as a function of different pH values.

Expt. # (pH)

| | 18 | 19 | 20 | 21 |
|---|---|---|---|---|
| Reagent | (10-10.5) | (9-9.5) | (8-8.5) | (7-7.5) |
| K₂[BH₃.CO₂] | 8.5 | 8.5 | 8.5 | 8.5 |
| (mg) | | | | |
| Na₂B₄O₇ | 1.9 | 1.9 | 1.9 | 1.9 |
| (mg) | | | | |
| NaF (mg) | 10.5 | 10.5 | 10.5 | 10.5 |
| NaH₂PO₄.H₂0 | 0 | 0 | 7.8 | 8.0 |
| (mg) | | | | |
| Na₂HPO₄ | 3.0 | 0 | 6.9 | 7.1 |
| (mg) | | | | |
| NaHCO₃ | 5.0 | 5.2 | 0 | 0 |
| (mg) | | | | |
| Na₂CO₃ (mg) | 0 | 4.0 | 0 | 0 |
| Yield (%) | 95 | 95 | 95 | 95 |

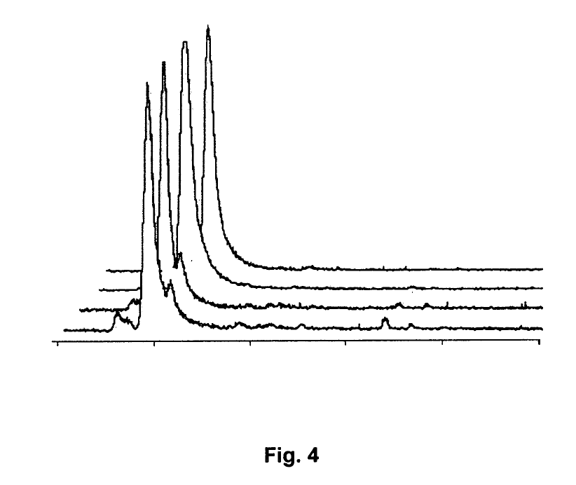

It has been demonstrated that for certain ligands, increasing the concentration of fluoride from 0.1 to 1.0 M improved the yields of rhenacarboranes so long as the ligands remain soluble. Since the method for ^{99m}Tc-metallocarborane formation involved essentially the same procedure, it seemed logical that higher quantities of fluoride ion would also improve the average yield of [^{99m}Tc(CO)₃(OH₂)₃]⁺. To test this hypothesis, a series of experiments were performed where the quantity of fluoride ion was increased incrementally (Table 3: entries 10-12; Table 4: entries 13-16), which as predicted, gave corresponding improvements to the yield of [^{99m}Tc(CO)₃(OH₂)₃]⁺. This is illustrated in FIG. 4, which shows the γ-HPLC radiochromatograms from experiments involving different amounts of fluoride.

Entry 11 (260 mM NaF), showed high conversion of [^{99m}TcO₄]⁻ to [^{99m}Tc(CO)₃(OH₂)₃]⁺, however some unidentifiable peaks appeared in the HPLC radiochromatogram (3.3 min, 5.5 min, and small peaks around 17 and 18 min). These peaks were not present for the reactions which employed 500 mM fluoride. Fluoride ion concentrations greater than this value produced the same results hence further increasing the amount of fluoride was unnecessary.

With the existing commercial kit, to investigate the influence of pH on the efficiency of labelling it is necessary to adjust the pH of the solution after the synthesis of [^{99m}Tc(CO)₃(OH₂)₃]⁺. A more convenient approach would be to design kits that would afford the ^{99m}Tc-trisaquaion at specific pH values directly. Besides convenience, this approach would also create the opportunity for “one pot” labelling procedures for biomolecules that are sensitive to high pH. To date however, [^{99m}Tc(CO)₃(OH₂)₃]⁺ has only been prepared under highly basic reaction conditions. With the stabilizing influence of fluoride being apparent, attempts were made to prepare [^{99m}Tc(Co)₃(OH₂)₃]⁺ directly at various pH values.

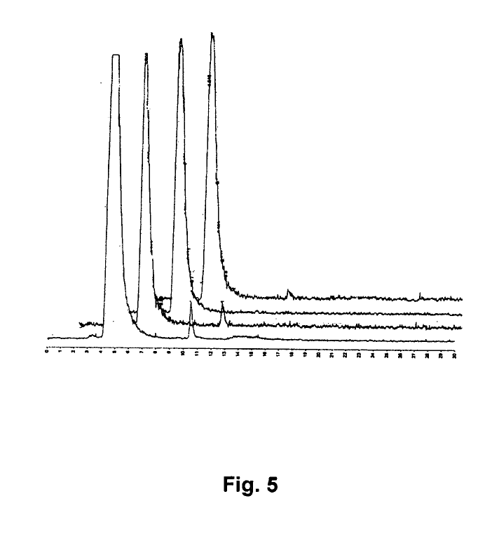

A series of reactions were performed in different buffers were the pH of the mixtures were tested prior to and after formation of 22b. This approach led to the successful development of several unique formulations for the preparation of [^{99m}Tc(CO)₃(OH₂)₃]⁺ at pH values ranging from 7 to 10.5. As illustrated in Table 5 (entries 18-21), the radiochemical yields of [^{99m}Tc(CO)₃(OH₂)₃]⁺ were unaffected by changes in pH. The γ-HPLC radiochromatograms from these entries are shown in FIG. 5. This set of experiments represents the first report of the direct formation of [^{99m}Tc(CO)₃(OH₂)₃]⁺ from conditions that are not highly alkaline (pH≧12) which makes developing one pot kits for pH sensitive biomolecules a real possibility.

One aspect that was not discussed was the influence of temperature. The formation of the [^{99m}Tc(CO)₃(OH₂)₃]⁺ cation using the commercial kit is normally achieved at 100° C. In the experiments described above, heating to reflux resulted in poor yields of the ^{99m}Tc-trisaquaion. The optimal temperature for the fluoride mediated formation of [^{99m}Tc(CO)₃(OH₂)₃]⁺ was between 65 and 70° C. Presumably, the stabilizing influence of fluoride ion is diminished at temperatures above 70° C. leading to more rapid decomposition of [^{99m}Tc(CO)₃(OH₂)₃]⁺.

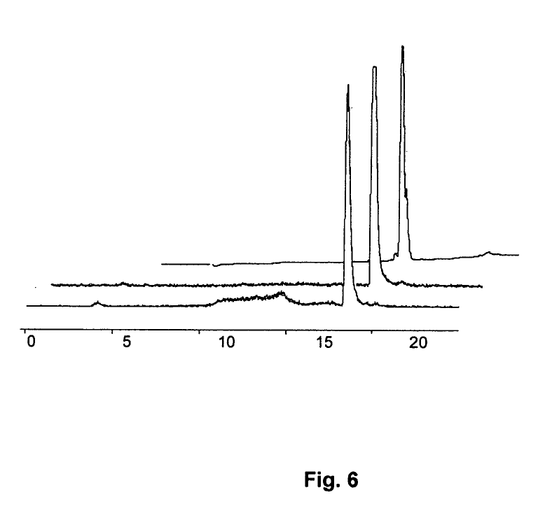

After establishing the necessary parameters needed to produce [⁹⁹Tc(CO)₃(OH₂)₃]⁺ in high yield, the synthesis of ^{99m}Tc-metallocarboranes was investigated. The initial experiments were performed with compound 1b (Scheme 11) because the carborane is soluble in aqueous solutions and because the Re and ⁹⁹Tc-analogues which were prepared previously could be used as well characterized reference standards. [^{99m}Tc(CO)₃(OH₂)₃]⁺ was prepared from 185-740 MBq (5-20 mCi) of [^{99m}TcO₄]⁻, using the optimal conditions described above. Compound 1b, which had been pre-incubated with fluoride ion, was subsequently added directly into the reaction vial. The temperature was raised to 85° C. from the initial 70° C. used to prepare [^{99m}Tc(CO)₃(OH₂)₃]⁺, and the reaction progress monitored by HPLC (FIG. 6). After 3 hours the reaction was complete with the major product being the Tc-carborane with the minor impurities being [^{99m}Tc(CO)₃(OH₂)₃]⁺ and [^{99m}TcO₄]⁻.

The next step involved separating 23b from residual [^{99m}Tc(CO)₃(OH₂)₃]⁺, Na[^{99m}TcO₄], and unreacted ligand. This could have been performed by semi-preparative HPLC however this approach is time consuming and impractical for routine clinical use. A convenient alternative involved solid-phase extraction using a commercially available C₁₈ Sep-Pak® cartridge. In the procedure described here, it was necessary to first condition the Sep-Pak® with acetonitrile, ethanol, and 10 mM HCl. The entire crude reaction mixture was then loaded onto the column and eluted with 10 mM HCl. Residual [^{99m}TcO₄]⁻ and [^{99m}Tc(CO)₃(OH₂)₃]⁺ are both removed under these conditions, while the target complex remains on the Sep-Pak®. After elution with acetonitrile, the product was obtained in greater than 99% purity (FIG. 6).

One important feature of this purification protocol is that it results in the removal of excess ligand giving the product in high effective specific activity. The importance of this result lies in the fact that removal of residual ligand from the purified product assists in the prevention of unwanted biological effects once the tracer is administered. Furthermore, if a ligand and its ^{99m}Tc-complex have similar affinities for a particular receptor, then the excess unlabelled ligand can prevent binding of the ^{99m}Tc-labelled compound to the target.

Another important criterion that needed to be evaluated was stability. In vivo, there are many endogenous ligands that can degrade ^{99m}Tc complexes through transmetalation reactions (K. Schwochau Angew. Chem. Int. Ed. 1994, 33, 2258). To test the stability of the ^{99m}Tc-metallocarborane towards ligand exchange, compound 23b was incubated with a 1000-fold excess of cysteine and histidine in separate experiments. After incubation at 37° C. in phosphate buffered saline (PBS, pH=7.2) for 24 hours, the γ-radiochromatograms from both experiments indicated greater than 99% of the product remained intact. The stability of the complexes supports the potential use of ^{99m}Tc-metallocarboranes as synthons for preparing radiopharmaceuticals.

To study the factors that impact the yield of 23b, pH, ligand concentration, and fluoride ion concentration, were systematically varied. With the exception of the ligand concentration, these adjustments were made to the kit used to prepare [^{99m}Tc(CO)₃(OH₂)₃]⁺, taking advantage of the initial work on developing different fluoride kits, which allow for direct control of pH and fluoride ion concentration.

The mild reaction conditions afforded by the fluoride-based method for the preparation of the rhenacarboranes cannot necessarily be directly correlated to chemistry at the tracer level. The discrepancy lies in the fact that at the tracer level, [^{99m}Tc(CO)₃(OH₂)₃]⁺ is normally prepared under highly basic conditions whereas, the corresponding rhenacarboranes are prepared effectively at neutral pH. Since the initial method used to prepare ^{99m}Tc-metallocarboranes involved direct incubation of the nido-carborane ligand with [^{99m}Tc(CO)₃(OH₂)₃]⁺ without any adjustment of pH, the question remained as to whether fluoride mediates the metallation reaction or whether product formation was a consequence of the high pH.

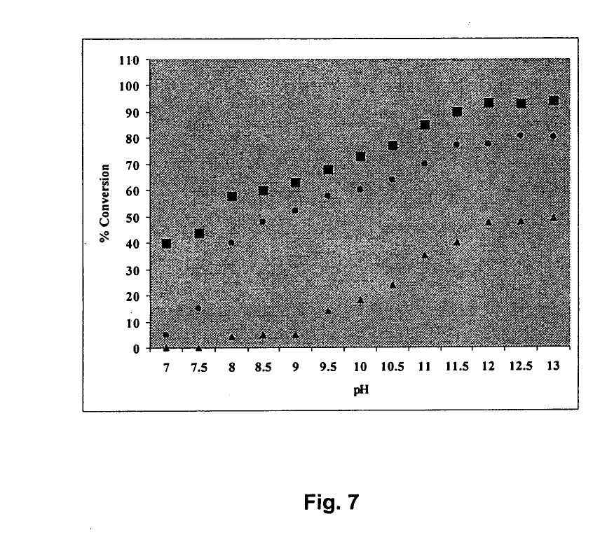

To investigate the impact of basicity, labelling reactions were performed at various pH values using a fixed fluoride ion concentration of 0.1 M and reaction time of 3 hours. As FIG. 7 illustrates, the yield of 23b increases with increasing pH and that a high pH is needed to afford good yields of the ^{99m}Tc-metallocarborane, which is not the case for rhenium at the macroscopic level. The results do demonstrate that it is possible, albeit in reduced yields, to prepare metallocarboranes at low pH, which will be important in cases where base sensitive targeting vectors are attached to the cluster. These results also indicate that fluoride ion is clearly involved in mediating the formation of 23b since a yield of 40% was observed at neutral pH at a ligand concentration of 10⁻² M. In the absence of fluoride under the same conditions, no product was detected.

FIG. 7 also summarizes the results of a series of experiments that were performed to evaluate the effect of changing the concentration of the ligand on the yield of the Tc-metallocarborane. The results clearly demonstrate that the concentration of the ligand does play a significant role since the percent conversion roughly doubles when the ligand concentration is increased to 10⁻² M from ₁₀⁻⁴ M. The importance of a low ligand concentration for preparations that do not involve further purification lies predominantly in minimizing side effects and/or receptor saturation once the radiotracer is administered in vivo. In this respect, both the Cp⁻ and the [nido-7,8-C₂B₉H₁₂]⁻ anion are less efficient than the bi- and tridentate chelates for Tc(I) where Schibli and coworkers have demonstrated that mild radiolabelling conditions (30 min, 75° C.) can be used to prepare complexes of [^{99m}Tc(CO)₃(OH₂)₃]⁺ with tridentate chelates in yields of ≧95% at ligand concentrations ranging from 10⁻⁴ to 10⁻⁶ M (Schibli, R.; Bella, R. L.; Alberto, R.; Garcia-Garayoa, E.; Ortner, K.; Abram, U.; Schubiger, P. A. Bioconjugate Chem. 2000, 11, 345). In the case of the AcCp system, the concentration of the ligand used was 10⁻³ to 10⁻⁴ M. Although carborane concentrations of 10⁻³ to 10⁻⁴ M do not produce as high a yield as the AcCp system, this limitation is overcome since excess ligand can readily be removed using the simple purification method described above. The general utility of this approach for purifying other carboranes requires further investigation.

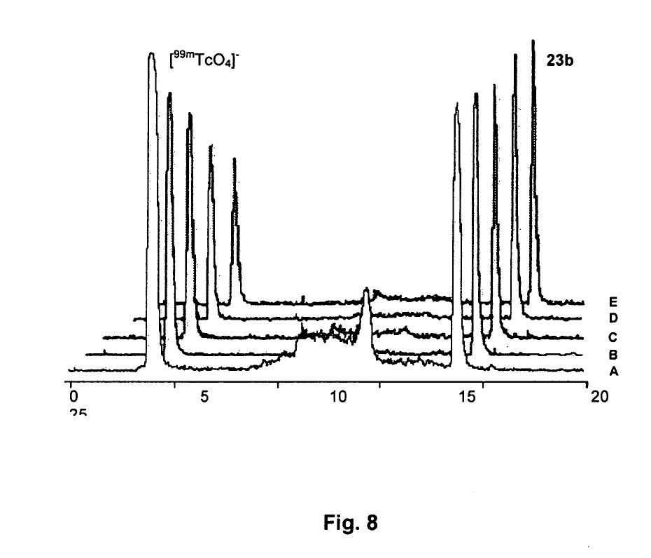

Another important factor that was investigated was the impact of changing the fluoride ion concentration on the yield of ^{99m}Tc-metallocarborane formation. A series of experiments were performed where [^{99m}Tc(CO)₃(OH₂)₃]⁺ was prepared in the presence of varying amounts of fluoride ion (10, 100, 500, and 1000 mM NaF) followed by the addition of compound 1b. The progress of the reaction was monitored by HPLC (FIG. 8), which showed that increasing the fluoride ion concentration increases the yield of the product. This is consistent with the results observed for rhenium.

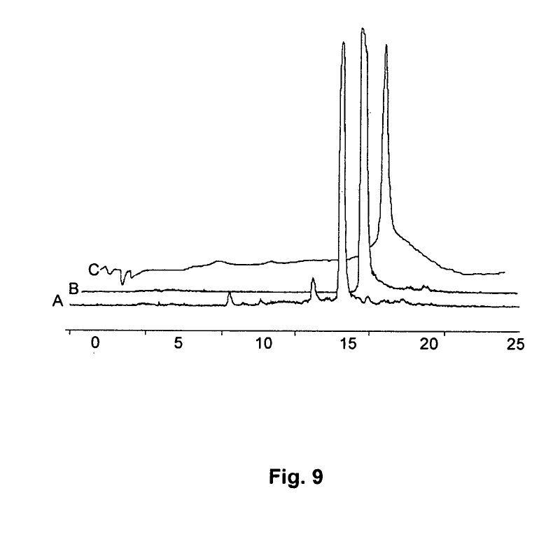

As shown in Scheme 2, compound 4 (At a ligand concentration of 10⁻³ M) was combined with [^{99m}Tc(CO)₃(OH₂)₃]⁺ (85° C., pH 12) in 0.5 M fluoride. Under these conditions, nearly quantitative conversion of 4 to the desired product 5 was observed in the HPLC radiochromatogram in approximately half of the reaction time (1.5 hours) required to achieve the maximum yield of compound 23b (FIG. 9). Afterwards, the Sep-Pak® purification protocol described above was employed to isolate compound 5 in 70% yield free from residual ligand. Cysteine and histidine challenge experiments were performed under the same conditions used for 23b where there was no sign of decomposition.

In summary, a novel strategy for the preparation of Re, ^{99m}Tc and other metallocarboranes in water under mild reaction conditions has been developed. The reported Tc complexes are attractive synthons for the preparation of organometallic radiopharmaceuticals because of their inertness, relatively small size, and ease of derivatization. Furthermore, with the hard base reaction in hand, it is now possible to use the numerous carborane derivatives that have been designed to target tumors for boron neutron capture therapy as the basis for designing novel ^{99m}Tc radiopharmaceuticals (Soloway, A. H.; Tjarks, W.; Barnum, A.; Rong, F.-G.; Barth, R. F.; Codogni, I. M.; Wilson, J. G. Chem. ReV. 1998, 98, 1515).

A synthetic strategy was used to prepare metal complexes of a wide variety of both ortho and meta carborane derivatives. It was also shown that metallocarboranes can be prepared from both nido-carboranes and closo-carboranes. The fluoride-based approach is mild compared to traditional synthetic methods and should be adaptable for the preparation of the corresponding ^{99m}Tc-carborane complexes.

It was also shown that aqueous fluoride can be used to facilitate the preparation of [^{99m}Tc(CO)₃(OH₂)₃]⁺ and Tc-labeled carboranes. Simple formulations were developed that allow for the synthesis of [^{99m}Tc(CO)₃(OH₂)₃]⁺ at specific pH values including those that are much less basic than what is produced using the commercially available kit. In terms of the radiosynthesis of ^{99m}Tc-metallocarboranes, a small collection of ligands were successfully labeled and the products isolated free from any unreacted ligand. The products displayed resistance to ligand exchange by both cysteine and histidine. It was determined that a number of factors, most notably ligand and fluoride concentrations and pH influence the overall yield of the reaction. Having the ability to label carboranes with Tc creates the means to explore the possibility of using the substantial number of targeted carborane derivatives that have been developed for boron neutron capture therapy ((a) Soloway, A. H.; Tjarks, W.; Barnum, A.; Rong, F.-G.; Barth, R. F.; Codogni, I. M.; Wilson, J. G. Chem. Rev. 1998, 98, 1515 (b) Valliant, J. F.; Guenther, K. J.; King, A. S.; Morel, P.; Schaffer, P.; Sogbein, O. O.; Stephenson, A. Coord. Chem. Rev. 2002, 232, 173) as the basis for designing novel radiopharmaceuticals for imaging tumours. Furthermore, it may also be possible to use fluoride to generate “one pot” kits for preparing Tc(I) radiopharmaceuticals and for improving the labelling yields of other ligand systems and bioconjugates, which did not show satisfactory conversion to the desired products when reactions were carried out using the existing commercial kit.

The following non-limiting examples are illustrative of the present invention:

### EXAMPLES

Materials and Instrumentations: Potassium boranocarbonate (K₂H₃BCO₂) was prepared following literature procedures (Alberto, R.; Ortner, K.; Wheatly, N.; Schibli, R.; Schubiger, A. P.; J. Am. Chem. Soc. 2001, 123, 3135). Analytical TLC's were performed on silica gel 60-F254 (Merck) and boron compounds were visualized with 0.1% PdCl₂ in hydrochloric acid (3.0 M), which upon heating gave dark brown spots. NMR spectroscopy was performed on a Bruker Avance AV600 spectrometer at ambient temperature. The chemical shifts (δ) for ¹H and ¹³C were recorded relative to solvent peaks as internal standards. BF₃-Et₂O was used as the reference standard for ¹¹B spectra. Electrospray ionization (ESI) mass spectrometry experiments were performed on a Micromass Quattro Ultima instrument where samples were dissolved in 1:1 CH₃OH/ H₂O or 1:1 CH₃CN/H₂O mixtures. High resolution MS was obtained using FAB MS and a Waters-Micromass Q-TOF Ultima Global instrument. IR spectra were run on a Bio-Rad FTS-40 FTIR spectrometer. HPLC experiments were performed on a Varian Prostar Model 230 instrument, fitted with a Varian Pro Star model 330 PDA detector and an IN/US γ-RAM gamma detector. The wavelength for detection was set at λ=254 nm and the dwell time in the gamma detector was 1 s in a 10 μL loop. All runs were performed using a Varian DYNAMAX (250 mm×4.6 mm), MicroSorb-MV analytical column (300 Å-5 μm, RP-C18). The elution conditions consisted of: Method A: Solvent A=0.1% TFA in H₂O, Solvent B=0.1% TFA in CH₃CN: Gradient Elution: 0-3 min, 100% A to 95% A; 3-6 min, 75% A; 6-9 min, 66% A; 9-20 min, 0% A; 20-22 min, 0% A; 22-24 min, 95% A; 24-25 min, 100% A. Method B (which was used for the histidine and cysteine challenge experiments): Solvent A=H₂O, Solvent B=CH₃CN: Gradient Elution: same as above. Method C: Solvent A=tetraethylammonuium phosphate (TEAP; pH=2-2.5), Solvent B=CH₃OH: Gradient Elution: 0-3 min, 100% A; 3-6 min, 100% A to 75% A; 6-9 min, 75% A to 67% A; 9-20 min, 67% A to 0% A; 20-22 min, 0% A; 22-25 min, 0% A to 100% A; 25-30 min, 100% A. The flow rate for all methods was set at 1 mL/min. Caution: ^{99m}Tc is a remitter (141 KeV) with a half-life of 6 hours, which should only be handled in appropiately shielded and licensed laboratories.

### Example 1

### Synthesis of 2a

In two separate vials compound 1a (0.11 g, 0.64 mmol) and [NEt₄]₂[Re(CO)₃Br₃] (0.49 g, 0.64 mmol) were suspended in 100 mM solution of aqueous KF (5.0 mL) and heated to 85° C. under Ar. After one hour, the solution containing [NEt₄]₂[Re(CO)₃Br₃] was added dropwise to the solution containing the nido-carborane while maintaining the temperature at 85° C. After 3.5 hours, the heterogeneous suspension became clear and the reaction was allowed to proceed at 85° C. for 18 hours. After cooling the clear yellow solution to room temperature a precipitate formed which was collected by filtration. The desired product was isolated by preparative TLC (92:8 CH₂Cl₂/CH₃OH+0.1% AcOH). TLC analysis indicated additional product remained in the aqueous layer. Consequently, the filtrate was concentrated under reduced pressure, and the resulting colourless solid washed with THF. An additional aliquot of sample from the THF solution was isolated by preparative TLC. Yield: 0.12 g, 34%; TLC Rf (17:3 CH₂Cl₂/CH₃OH+0.1 % AcOH)=0.45; ¹H NMR (acetonitrile-d₃, 600.13 MHz): δ 0.5-1.7 (br, BH), 1.29 (br, CH₃), 1.94 (br, CH₂), 2.34 (br, CH); ¹³C{¹H} NMR (acetonitrile-d₃, 150.92 MHz): δ 33.53, 40.72, 55.0, 199.47; ¹¹B{¹H} NMR (CD₂Cl₂/acetonitrile-d₃+1 drop of DMSO-d₆, 160.46 MHz): δ 6.86, −10.50, −14.71, −21.73, −23.12; FTIR (KBr, cm⁻¹): 3048 (s), 2924 (s), 2558 (s), 2510 (s), 1893 (s); MS (ESI): m/z 403.1 [M⁻]; HRMS m/z calculated for C₅B₉H₁₁O₃Re: 403.1157 gmol⁻¹; Observed: 403.1159.

### Example 2

### Synthesis of 2b

In two separate vials compound 1b (0.065 g, 0.23 mmol) and [NEt₄]₂[Re(CO)₃Br₃] (0.18 g, 0.23 mmol) were suspended in a 100 mM solution of aqueous KF (2.5 mL) and heated to 85° C. under Ar. After one hour the solution containing the [NEt₄]₂[Re(CO)₃Br₃] was added dropwise to the solution containing the nido-carborane while maintaining the temperature at 85° C. After 30 minutes, the heterogeneous mixture became clear and the reaction was allowed to proceed at 85° C. overnight. After cooling to room temperature, the pH was adjusted to 3 using 12 M HCl. The aqueous solution was then passed through a fritted funnel containing Celite yielding a brown solid upon concentration of the filtrate under reduced pressure. The colourless product was purified by flash column chromatography (gradient elution: 19:1 CH₂Cl₂/CH₃OH to 4:1 CH₂Cl₂/CH₃OH) on silica gel (0.068 g, 50%). TLC Rf (4:1 CH₂Cl₂/CH₃OH)=0.44; ¹H NMR (CD₃OD, 600.13 MHz): δ 1.27 (t, J=7.2 Hz, CH₃), 2.07 (m, CH₂CH₂CO₂H), 2.29 (m, CH₂CH₂CO₂H), 3.27 (t, NCH₂); ¹³C{¹H} NMR (CD₃OD, 150.92 MHz): δ 7.59, 29.04, 35.27, 36.50, 42.44, 46.21, 53.25, 176.89, 199.81, 200.66; ¹¹B{¹H} NMR (CD₃OD, 160.46 MHz): δ −10.89, −13.79, −16.87, −18.24, −21.82, −33.151, −37.04; FTIR (KBr, cm⁻¹): 3620 (w), 2928 (m), 2531 (s), 2007 (s), 1907 (s); MS (ESI): m/z 474.71 [M⁻]; HRMS m/z calculated for C₈H₁₅ B₉O₅Re: 475.1299 gmo⁻¹; Observed 475.1302; HPLC (method A): t_{R}=18.3 min.

### Example 3

### Synthesis of 2c

Compound 1c (0.15 g, 0.70 mmol) was dissolved in a 1:1 mixture of aqueous 100 mM KF and methanol (10 mL) and the temperature elevated to 85° C. for 1 hour. [NEt₄]₂[Re(CO)₃Br₃] (0.54 g, 0.70 mmol) was dissolved in distilled de-ionized water and the temperature elevated to 85° C. under Ar(g). After 1 hour, the solution containing 1c was added to the solution containing [NEt₄]₂[Re(CO)₃Br₃] and the reaction heated to reflux. After 13 hours, the reaction was allowed to cool to room temperature. The resulting heterogeneous solution was acidified with 1 M HCl (3 mL) and subsequently extracted with 50 mL of ethyl acetate. The ethyl acetate was extracted with 1 M HCl (2×50 mL), dried over anhydrous MgSO₄ and concentrated under reduced pressure giving an off-white solid. The solid was re-dissolved in CH₃CN/H₂O (2:3 v/v mixture, 10 mL) and purified using gel permeation chromatography (Sephadex G-10; column volume of 12.5 cm×1.5 cm; flow rate of 1.5 mL/min) yielding a white solid (0.24 g, 70 %). TLC Rf (17:3 CH₂Cl₂/CH₃OH+0.1% AcOH)=0.33; m.p.: 240° C. (decomp.); ¹H NMR (acetoned₆, CD₃OD, 600.13 MHz): δ 1.52 (m, CH₂CH₂CH₂), 1.54 (s, CH), 1.66 (m, CH₂CH₂CH₂), 1.94 (m, CH₂(CH₂)₂N), 3.15 (m, N(CH₃)₂), 3.23 (m, (CH₂)₂CH₂NH), 5.75 (s, NH); ¹³C{¹H} NMR (acetone-d₆, 150.92 MHz): δ 26.92, 36.67, 44.14, 59.41, 199.68; ¹¹B{¹H} NMR (acetone-d₆, 192.54 MHz): δ −10.22, −10.75, −13.37, −15.26, −18.69, −21.04, −32.53, −36.43.; IR (KBr, cm⁻¹): 3446 (w), 3148 (w), 2526 (s), 2006 (s), 1899 (s); HRMS (FAB): m/z calculated for C₁₀H₂₃B₉NO₃Re: 487.2121 gmol-1; Observed 487.2114.

### Example 4

### Synthesis of 2d

A penicillin vial (10 mL) containing freshly prepared potassium boranocarbonate (K₂H₃BCO₂), (8.5 mg, 62.5 μmol), Na₂B₄O₇.10H₂O (2.9 mg, 7.6 μmol), KF (2.8 mg, 48.2 μmol) and Na₂CO₃ (4.0 mg, 37.7 μmol) was capped with a rubber stopper and flushed with N₂(g) for 45 minutes. ^{99m}TcO₄⁻ (10-20 mCi, 370-740 MBq) in 500 μL of saline was added via a syringe, and the mixture slowly heated to 70° C. over a period of 1 hour. After cooling in an ice bath, an aliquot was taken to verify formation of [Tc(CO)₃(OH₂)₃]⁺. HPLC (method A): t_{R}=12.5 min; Yield ≧95%. In a separate penicillin vial, K[C₂B₉H₁₂(CH₂)₂CO₂K] (5.0 mg, 17.6 μmol) was suspended in 250 μL of a 100 mM de-gassed aqueous solution of KF and the vial capped and flushed with N₂. The mixture was heated at 85° C. for one hour prior to adding the solution containing the ligand to [^{99m}Tc(CO)₃(OH₂)₃]⁺ via syringe. After combining the solutions the temperature was raised to 85° C. for 3 hours. The reaction was subsequently cooled on an ice-bath, loaded onto a pre-conditioned Sep-Pak, which was eluted slowly with 10 mM HCl (7 mL), 1:1 acetonitrile/10 mM HCl (2 mL), and finally acetonitrile (10 mL). The product, which was in the acetonitrile washing, was isolated in 80% radiochemical yield in >99% purity as determined by analytical HPLC. HPLC (method A): t_{R}=18.4 min.

### Example 5

### Synthesis of 4

Compound 3 (0.10 g, 0.434 mmol) and powdered potassium hydroxide (0.20 g, 3.4 mmol) were combined in a 95:5 (v/v) mixture of absolute ethanol/ddH₂O (8 mL) at room temperature under N₂(g). The suspension was heated to 90° C. for 12 hours, cooled to room temperature and CO₂(g) bubbled through the homogeneous solution to precipitate the excess KOH as K₂CO₃. The heterogeneous mixture was passed through a fritted funnel packed with Celite, which was subsequently washed with absolute ethanol (3×10 mL). After concentration of the filtrate under reduced pressure, the mixture was purified by flash chromatography through silica gel (isochratic elution: 1:9 CH₃OH/CH₂Cl₂+0.1% AcOH). The oily residue was suspended in ddH₂O (10 mL) and lyophilized at −80° C. to yield a white solid (0.049 g, 50%). TLC R_{f} (85:15 CH₂Cl₂/CH₃OH+0.1% AcOH)=0.28; ¹H NMR (500.13 MHz, 5:1 D₂O/CD₃OD): δ −0.5 −2.8 (bm, BH), 0.94 (m, CH₂), 1.08 (m, CH₂); ¹³C{¹H} NMR (50.3 MHz, CD₃OD): δ 18.27, 23.47, 32.35, 58.28, 180.44; ¹¹B{¹H} NMR (160.5 MHz, CD₃OD): δ −5.14, −6.41, −22.20, −23.19, −35.11, −36.14; FTIR (KBr, cm⁻¹): ν 3220, 2533, 1429; ESMS (negative ion): 205.48 [M-H]⁻.

### Example 6

### Synthesis of 2b from the closo-carborane

Compound 6 (0.050 g, 0.231 mmol) and [NEt₄]₂[Re(CO)₃Br₃] (0.196 g, 0.254 mmol) were combined in a 10 mL penicillin vial, sealed with rubber septum and aluminum cap, and then flushed with N₂(g) for 10 minutes. A solution containing 500 mM TEAF(aq)/absolute EtOH (9:1 v/v) was added (1.0 mL) and the resultant suspension heated to 100° C. After 30 hours, the heat was removed and the mixture acidified by the addition of 12 M HCl. CH₃CN was subsequently added (1.0 mL) and the vial vigorously shaken for 5 minutes. The mixture was frozen at −5° C. overnight in a freezer resulting in a biphasic mixture with the organic layer portioned on top of the frozen aqueous layer. The organic layer was decanted and concentrated yielding a brown viscous oil. The product was isolated by flash column chromatography through silica gel (gradient elution: 95:5 CH₂Cl₂/CH₃OH to 90:10 CH₂Cl₂/CH₃OH) as a cream coloured solid upon concentration under reduced pressure (0.098 g, 70%). TLC R_{f} (4:1 CH₂Cl₂/CH₃OH)=0.44; mp>140° C. (decomp.); ¹H NMR (300.13 MHz, acetone-d₆): δ 1.15 (m, CH₃), 1.81 (m, CH₂), 2.33 (m, CH₂), 3.23 (t, CH₃); ¹³C{¹H} NMR (151 MHz, acetone-d₆): δ 7.59, 29.04, 35.27, 36.50, 37.03, 42.44, 46.21, 53.25, 176.89, 199.81, 200.66; ¹¹B{¹H} NMR (160.5 MHz, acetone-d₆): δ 18.82, −5.79, −8.13, −10.46, −11.87, −18.56, −20.02, −22.17; FTIR (KBr, cm⁻¹): ν 2542, 1998, 1893; HRMS (ESI-Q-TOF): Calcd for B₉C₈H₁₅O₅Re: 474.7085. Found: 475.1302.

### Example 7

### Synthesis of 9

Compound 8 (0.100 g, 0.426 mmol) was combined with potassium hydroxide (100 mg, 1.42 mmol) and dissolved in absolute ethanol (2.5 mL). The reaction mixture was heated at reflux for 24 hrs, the temperature lowered to room temperature and CO₂(g) passed through the solution, resulting in the formation of a thick white precipitate. The heterogeneous mixture was filtered, and the clear, colorless eluent concentrated in vacuo giving a viscous, opaque oil. The crude oil was dissolved in distilled, deionized water (3 mL) and lyophilized giving the product as a white solid (>99%). TLC R_{f} (22% MeOH in CH₂Cl₂)=0.56; mp>225° C. (decomp.); ¹H NMR (600 MHz, CD₃OD): δ 8.37 (d, J=4.59, 1H, H-5), 7.71 (m, 1H, H-3), 7.25 (d, J=7.51, 1H, H-2), 7.14 (m, 1H, H-4), 3.68 (bs, carborane CH), 3.036 (AB, J=−15.3, 1H, CH₂), 2.78 (AB, 1H, CH₂), 0-2.7 (br m, BH); ¹³C{¹H}NMR (151 MHz, CD₃OD): δ 162.0, 146.53, 136.49, 123.22, 120.63, 57.9, 46.53, 44.90; ¹¹B{¹H} NMR (160.5 MHz, CD₃OD): δ −8.89, −9.96, −11.96, −14.40, −17.29, −18.25, −19.90, −31.86, −35.44; FTIR (KBr, cm⁻¹): ν 2990, 2940, 2517, 1749; HRMS (ESMS-QTOF) calcd for C₈H₁₇B₉N: 225.2241. Found: 225.2246.

### Example 8

### X-Ray Crystallography of 2c

X-ray diffraction data for compound 2c was collected on a single crystal grown from a CH₃OH/CH₂Cl₂ mixture (1:1 v/v) (0.07×0.06×0.01 mm³). Data was collected on a p4 Bruker diffractometer fitted with a rotating anode, a Bruker SMART-1K CCD (charge coupled device) area detector and an Oxford Cryostream cooling system. Diffraction data was collected using the program SMART with graphite-monochromated Mo—K_{α} X-radiation (ν=0.71073 Å) and a single crystal of 2c mounted on the tip of a glass fibre. The crystal to detector distance was 4.987 cm. Initially, accurate unit cell parameters were determined at 173 K with better than 0.9 Å resolution from a least squares fit of the strong reflections, collected by a 12° scan in 40 frames using the SMART software. Data were obtained from a chosen number of centered reflections of the setting angles (χ, φ, and 2θ) in reciprocal space with truncation of the data (2θ=48°), using least squares, due to disorder in the high angle reflections. Data reduction was carried out using the SAINT program applying polarization and Lorentz corrections to the integrated diffraction spots. The raw frame data and the structure was solved from direct methods and refined by full-matrix least squares on F² using the Bruker SHELXTL PLUS package. Corrections were made for decay and an empirical absorption correction was made with SADABS program based on redundant reflections. Additionally, after completion of data collection, the first 50 frames were re-acquired for the improvement of the decay correction. All non-hydrogen atoms were refined with anisotropic atomic displacement parameters giving rise to the prescribed R₁ values except for the carbonyl atoms (C₁, C₂, C₃, O₁, O₂, and O₃), which were refined isotropically due to positional disorder. All hydrogen atoms were assigned based on the difference map and added as fixed contributors at calculated points with isotropic thermal parameters based on their respective carbon atoms. Crystallographic data is presented in Table 1. The structure is shown in FIG. 3.

### Example 9

### Synthesis of 10

Compound 9 (11.4 mg, 0.044 mmol) and [NEt₄]₂[Re(CO)₃Br₃] (32 mg, 0.042 mmol) were combined and dissolved in 100 mM aqueous potassium fluoride (2 mL) and the mixture heated to reflux for 24 hrs. Upon cooling to room temperature, the pH was adjusted using 0.1 M HCl (final pH ˜1) and the mixture extracted with CH₂Cl₂ (3×10 mL). All organic portions were combined, dried over sodium sulphate, and the solvent removed by rotary evaporation giving pure 10 (19 mg, 85%), as an off-white semi-solid. No further purification was required. TLC R_{f} (18% MeOH in CH₂Cl₂)=0.05; ¹H NMR (600 MHz, CD₃OD): δ 8.57 (d, H-5), 7.91 (m, 1H, H-3), 7.41 (m, 2H, H-2, H-4), 3.69 (bs, carborane CH), 3.31 (m, CH₂), 2.1-1.01 (bm, BH); ¹³C{¹H} NMR (50.3 MHz, CD₃OD): δ 199.90, 155.32, 146.38, 142.49, 130.32, 126.43, 50.37, 45.17, 29.25; ¹¹B{¹H}NMR (192.54 MHz, CD₃OD) δ −7.88, 11.44, 14.11, 16.77, 18.20, 19.96; IR (KBr, cm⁻¹): ν 2546, 1993, 1885, 1626; HRMS (ESMS-QTOF) Calcd for C₁₁H₁₆O₃B₉NRe: 494.1581. Found: 494.1584.

### Example 10

### Synthesis of 11

1-(3′-Chloropropyl)-1,2-dicarba-closo-dodecaborane (1.23 g, 5.57 mmol) and sodium iodide (1.67 g, 11.14 mmol) were combined in dry acetone (150 mL) under nitrogen and heated to reflux for 22 hours. A precipitate appeared, which upon completion of the reaction, was collected by filtration through a medium porosity fritted funnel. The residue was washed with diethyl ether (3×25 mL) and all organic fractions pooled and concentrated under reduced pressure giving an off-white solid. The solid was subsequently dissolved in diethyl ether (25 mL), which was extracted with 0.1 M sodium thiosulfate (2×25 mL). The aqueous layer was further extracted with ether (2×20 mL) and the organic fractions combined, dried over Na₂SO₄, filtered and the filtrate concentrated to dryness under reduced pressure yielding a white solid. The product was purified by flash column chromatography (isocratic elution,: 100% CHCl₃) through silica gel to give a white solid (1.42 g, 81%). TLC R_{f} (CHCl₃)=0.56; ¹H NMR (200.13 MHz, CDCl₃): δ 0.8-3.6 (bm, BH), 1.91 (m, CH₂), 2.33 (m, CH₂), 3.08 (t, CH₂), 3.51 (bs, carborane CH); ¹³C{¹H} NMR (50.3 MHz, CDCl₃): δ 3.61, 32.33, 38.84, 61.50, 73.77; ¹¹B{¹H} NMR (160.5 MHz, CDCl₃): δ −2.65, −6.05, −9.64, −12.12, −12.52, −13.46; FTIR (KBr, cm⁻¹): ν 2959, 2591; MS (El): m/z=312 [M]⁺, 183 [M-I]⁺.

### Example 11

### Synthesis of 13a

Compound 12 (0.506 9, 1.00 mmol) and KOH (1.2 g, 22 mmol,) were dissolved in absolute ethanol (20 mL) and the mixture heated to overnight. The reaction was cooled to room temperature and the excess KOH was precipitated as K₂CO₃ by passing a stream of CO₂ gas through the solution. The solid was removed by filtration and the residue washed with cold ethanol (20 mL). The combined filtrates were concentrated under reduced pressure yielding a white solid, which was dissolved in distilled water, and the pH adjusted to approximately 4 by the dropwise addition of 1M HCl. The solution was again concentrated to a white solid by rotary evaporation. The product was purified by silica gel column chromatography using a gradient of 50% to 70% acetone in CH₂Cl₂. A white solid was obtained by evaporating ether solutions of the product fraction. Yield: 83% (0.3 g). TLC R_{f} (25% CH₃OH/CH₂Cl₂)=0.38; ¹H NMR (500 MHz, CD₃OD): δ 4.51 (d, H-1, ³J_{1,2}=7.8 Hz ), 4.41 (d, H-1⁻,³J_{1′,2′}=7.8 Hz), 4.01 (d, 1H), OCH₂C_{cage}C_{cage}H, ²J_{7a,7b}=−10.9 Hz, H-7a), 4.01 (2dd, H-6a,6a′), 3.88 (d, 1H, ²J_{7a′, 7b′}=−10.7 Hz, H-7a′), 3.86 (2dd, H-6b,6b′), 3.77 (d, H-7b′), 3.64 (d, H-7b), 3.54-3.48 (m, H-3,3′, H-4,4′), 3.40 (m, H-5,5′), 3.34 (m, H-2,2′), 2.07 (br s, OCH₂C_{cage}C_{cage}H, H-9,9′), 2.14-0.30 (br, m, B—H), −2.5 (br, B—H—B); ¹³C NMR (126 MHz, CD₃OD): δ 103.41, 103.01 (C-1,1′), 78.46 (C-7,7′), 77.12, 77.01 (C-3,3′), 77.84, 77.74 (C-5,5′), 75.16 (C-2,2′), 71.55 (C-4,4′), 62.51 (C-6,6′); ¹¹B NMR (160 MHz, CD₃OD): δ −-10.83, −16.72, −21.87, −33.02, −37.50; IR (KBr, cm⁻¹): ν 3429, 2526; HRMS (EI): Calcd for C₉H₂₄B₉O₆: 326.2455. Found: 326.2452.

### Example 12

### Synthesis of 13b

Compound 13a (0.103 g, 0.282 mmol) was dissolved in distilled deionized water (1 mL) and placed in a water/ice bath. Tetraethylammonium bromide in water (2.0 M; 141 μL, 0.282 mmol) was whereupon a white precipitate formed. After allow the precipitate to congeal, the solid was collected by vacuum filtration and dried using a lyophilizer. The product (0.075 g, 58%) was a white solid. TLC R_{f} (25% CH₃OH/ CH₂Cl₂)=0.54; ¹H NMR (500 MHz, acetone-d₆): δ 4.35 (d, 1 H, H-1, ³J_{1,2}=7.8 Hz), 4.25 (d, 1 H, H-1′, ³J_{1′,2′}=7.8 Hz), 3.84 (1H, OCHHC_{cage}C_{cage}H, H-7a, ²J_{7a,7b}=−10.9 Hz), 3.81 (2dd, 2H, H-6a, 6a′), 3.70 (d, 1H, OCHHC_{cage}C_{cage}H, H-7a′, ²J_{7a′,7b′}=−10.7 Hz), 3.71 (2dd, 2H, H-6b, 6b′), 3.59 (d, 1H, OCHHC_{cage}C_{cage}H, H-7b′), 3.49-3.40 (m, 3H, H-3,3′, OCHHC_{cage}C_{cage}H, H-7b), 3.46 (q, 8H, (CH₃CH₂)₄N⁺, ³J=Hz), 3.40 (m, 2H, H-4,4′), 3.26 (m, 2H, H-5,5′), 3.20 (m, 2H, H-2,2′), 1.90 (br s, 2H, OCH₂C_{cage}C_{cage}H, H-8,8′), 1.39 (tt, 12H, (CH₃CH₂)₄N⁺); ¹³C NMR (126 MHz, acetone-d₆): δ 103.07 (C-1), 102.58 (C-1′), 77.96, 77.85 (OCH₂C_{cage}C_{cage}H, C-7,7′), 77.43, 77.37 (C-3,3′), 77.28, 77.20 (C-5,5′), 74.85 (C-2,2′), 71.61 (C-4,4′), 62.70 (C-6,6′), 53.12 ((CH₃CH₂)₄N⁺), 7.76 ((CH₃CH₂)₄N^{+);} ¹¹B NMR (160 MHz, acetone-d₆): −8.86, −15.99, −21.01, −31.52, −35.85; FTIR (KBr, cm⁻¹): ν 3417 (s, br, O—H), 2526 (s, B—H); HRMS (EI): Calcd for C₉H₂₄B₉O₆: 326.2455. Found: 326.2462.

### Example 13

### Synthesis of 14

Compound 13b (0.21 g, 0.46 mmol), TEAF (0.35 g, 2.32 mmol), and [NEt₄]₂[Re(CO)₃Br₃] (0.432 g, 5.61 mmol) were dissolved in distilled water (10 mL) and the mixture heated to reflux for seven days. Analytical HPLC indicated complete consumption of the starting material and LC-MS indicated that the major peak in the chromatogram corresponded to the target mass. Semi-preparative HPLC (80:20 to 54:46 H₂O: AcN, t=20 min) was used to isolate the product. Yield: 45 mg (16%); ¹H NMR (600 MHz, CD₃CN): δ 4.19 (d, 1H, ³J_{1,2}=7.6 Hz, H-1), 3.90 (2d, 2H, ²J_{7a,7b}=−10.8 Hz, H-7a), 3.69 (dd, 1H, ²J_{6a,6b}=−11.5 Hz, H-6a), 3.56 (m, 2H, H-6b, 7b), 3.28 (pt, 1H, H-3), 3.22 (pt, 1H, H-4), 3.16 (q, m, NCH₂CH₃, H-5), 3.11 (pt, 1H, H-2), 1.81 (br s, 1H, OCH₂C_{cage}C_{cage}H, H-9), 1.21 (t, NCH₂CH₃); ¹³C NMR (151 MHz, CD₃CN): δ 200.39 (C≡O), 103.68 (C-1), 77.43 (C-3), 77.19 (C-5), 75.82 (C-7), 74.74 (C-4), 62.72 (C-6), 53.06 (NCH₂CH₃), 7.67 (NCH₂CH₃); ¹¹B{¹H} NMR (192 MHz, CD₃CN): δ −5.82, −7.65, −8.78, −11.62, −18.35, −19.55, −20.13; FTIR (KBr, cm⁻¹): ν 3425, 2537, 1999, 1898; HRMS (ES-QTOF): Calcd for C₁₂H₂₃B₉O₉Re: 595.1794. Found: 595.1785.

### Example 14

### Synthesis of 16

Aqueous sodium fluoride (500 mM, 5 mL) was added to compound 15 (50 mg, 0.21 mmol) along with 3 equivalents of [Re(CO)₃(OH₂)₃]Br (258 mg, 0.62 mmol) and the mixture heated to reflux for two days. After allowing the reaction to cool to room temperature, the mixture was acidified with 10 M HCl (5 mL). The solution was subsequently diluted with acetonitrile (10 mL) and cooled at −10° C. until the organic layer separated. The acetonitrile layer was removed by pipet and concentrated under reduced pressure. The product was isolated by silica gel chromatography (5% methanol/chloroform) as a dark brown oil (70 mg, 51%). TLC R_{f} (10% methanol/chloroform)=0.15; ¹H NMR (600 MHz, CD₃OD): δ 7.58 (d, 1H, H-aryl), 7.38 (d, 1H, H-aryl), 7.15 (m, 1H, H-aryl), 7.066 (d, J=7.8 Hz, 1H, H-aryl), 6.98 (t, J=7.8 Hz, 1H, H-aryl), 6.91 (d, J=15 Hz, 1H, H-aryl), 6.84-(t, J=7.8 Hz, 1H, H-aryl), 6.59 (d, J=12.2 Hz, 1H, H-aryl), 6.43 (d, J=8.4 Hz, 1H, H-aryl); ¹³C{¹H} NMR (151 MHz, CD₃OD): δ 206.01, 156.85, 154.73, 149.24, 144.42, 140.88, 135.56, 132.36, 130.66, 129.71, 128.20, 127.81, 127.22, 126.58, 125.71, 124.88, 114.57, 114.07, 58.54, 58.38, 57.11, 56.85; ¹¹B{¹H} NMR (160 MHz, CD₃OD): δ −9.04, −11.61, −15.80, 19.57, −20.46, −21.93; FTIR (KBr, cm⁻¹): ν 3440, 2557, 2001, 1900, 1615, 1513; ESMS (negative ion): 571.2 [M]⁻.

### Example 15

### Synthesis of 18

n-BuLi (2.77 mL, 6.93 mmol; 2.5 M in hexanes) was added dropwise to a rapidly stirring solution of 1,7-dicarba-closo-dodecaborane (compound 17) (1.0 g, 6.93 mmol) in dry diethyl ether (125 mL) at −10° C. under a nitrogen atmosphere. The reaction mixture, which was maintained at −10° C. for 45 minutes, was subsequently added dropwise over 15 minutes to a stirring solution of methyl 3-bromopropionate (823 μL, 1.27 g, 7.63 mmol) in dry diethyl ether (125 mL) at −10° C. under nitrogen. The temperature was maintained for an additional 30 minutes at −10°0 C. then brought to reflux. After 2.5 hours, the crude reaction was concentrated under reduced pressure yielding a viscous oil, which was re-suspended in diethyl ether (75 mL) and extracted with acidified brine (pH=0.1; 3×75 mL). The organic layer was dried over MgSO₄ and the solvent removed under reduced pressure giving a pale yellow oil, and the target was isolated by silica gel chromatography (gradient elution: 5% ethyl acetate to 10% ethyl acetate in hexanes). The desired product was recrystalized from a solution of CH₃CN/acetone (6:1) yielding a powdery white solid (0.76 g, 46%). TLC Rf (5:95 EtOAc/hexanes)=0.13; ¹H NMR (300.13 MHz, CDCl₃): δ 1.0-3.0 (bm, BH), 1.73 (m, CH₂), 2.44 (m, CH₂) 2.89 (s, CH₃), 3.52 (bs, carborane CH); ¹³C{¹H} NMR (50.3 MHz, CDCl₃): δ 28.04, 34.05, 41.56, 41.96, 80.53, 173.93; ¹¹B{¹H} NMR (96.3 MHz, CDCl₃,): δ −7.08, −10.93, −13.72, −17.41; FTIR (KBr, cm⁻¹): ν 3067, 2603, 1728; MS (ESMS): 273.3 [M+K]⁺.

### Example 16

### Synthesis of 7

Compound 18 (0.050 g, 0.217 mmol) and [NEt₄]₂[Re(CO)₃Br₃] (0.184 g, 0.234 mmol) were combined in a 10 mL penicillin vial, sealed with rubber septum and aluminum cap, and then flushed with N₂(g) for 10 minutes. A solution containing 500 mM TEAF(aq)/absolute EtOH (9:1 v/v) was added (1.0 mL) and the resultant suspension heated to 100° C. After 22 hours, the heat was removed and the mixture acidified by the addition of 12 M HCl. CH₃CN was subsequently added (1.0 mL) and the vial vigorously shaken for 5 minutes. The mixture was frozen at −5° C. overnight in a freezer resulting in a biphasic mixture with the organic layer portioned on top of the frozen aqueous layer. The organic layer was decanted and concentrated yielding a brown viscous oil. The product was isolated by flash column chromatography through silica gel (gradient elution: CH₂Cl₂ to 90:10 CH₂Cl₂/CH₃OH) as a cream coloured solid (0.061 g, 57%). TLC R_{f} (85:15 CH₂Cl₂/CH₃OH +0.1% AcOH)=0.44; ¹H NMR (500.13 MHz, 5:1 CD₃OD-acetone-d₆): δ 1.0-3.0 (b, BH), 1.39 (t, ³J=5.8 Hz, CH₃), 1.74 (bs, carborane CH), 2.09, 2.22 (t, CH₂), 2.33, 2.39 (m, CH₂) 3.48 (q, ³J=7.2 Hz, NCH₂); ¹³C{¹H} NMR (125.77 MHz, acetone-d₆): δ 7.47, 33.37, 37.59, 52.77, 53.69, 85.31, 172.73, 200.38; ¹¹B{¹H} NMR (160.46 MHz, CD₃OD): δ −6.68, −10.81, −13.06, −16.51, −18.44, −22.05; FTIR (KBr, cm⁻¹): ν 2032, 1915; HRMS (ESI-Q-TOF): Calcd for B₉C₉H₁₄O₅Re: 475.1370. Found: 475.1404.

### Example 17

### Synthesis of 19

n-BuLi (2.77 mL, 6.93 mmol, 2.5 M in hexanes) was added dropwise to a rapidly stirring solution of meta-carborane (1.0 g, 6.93 mmol) in dry diethyl ether (125 mL) at −10° C. under a nitrogen atmosphere. The reaction mixture, which was maintained at −10° C. for 45 minutes, was subsequently added dropwise over 15 minutes to a solution of di-benzyl-azodicarboxylate (DBZAD) (2.30 g, 7.7 mmol) in dry diethyl ether (125 mL) at −10° C. under nitrogen. The reaction was stirred for 1 hour, whereupon it was heated to reflux 2.5 hours. After cooling to room temperature, the reaction was concentrated under reduced pressure yielding a viscous oil, which was taken up in ethyl acetate (75 mL) and washed with 1.0 M HCl (3×75 mL). The organic layer was dried over MgSO₄ and the solvent removed under reduced pressure to yield a viscous oil, which was purified by chromatography on silica gel (isocratic elution: 1:4 Et₂O/p.Et₂O) giving the final product as a white solid (1.81 g, 59%). TLC R_{f} (3:7 Et₂O/p.Et₂O)=0.40; mp.=101-104° C.; ¹H NMR (300.13 MHz, CDCl₃): δ 0.9-3.3 (bm, BH), 2.88 (s, CH), 5.07-5.09 (m, CH₂), 6.65 (bs, NH), 7.18-7.30 (m, C6H₅); ¹³C{¹H} NMR (50.3 MHz, acetone-d₆): δ 35.49, 52.88, 68.20, 69.25, 128.25, 128.57, 134.69, 152.85, 155.01; ¹¹B{¹H} NMR (96.3 MHz, CDCl₃): δ −5.60, −11.45, −12.90, −15.65; FTIR (KBr): ν 3310, 3053, 2632, 1745; HRMS (ESI-Q-TOF): Calcd for C₁₈B₁₀H₂₆N₂O₄: 442.1265. Found: 441.1297.

### Example 18

### Synthesis of 20

Compound 19 (0.10 g, 0.23 mmol) and TEAF (0.15 g, 0.90 mmol) were suspended in wet THF (2.3 mL) and the mixture heated to 80° C. for 18 hours. The mixture was subsequently cooled to room temperature, concentrated to dryness and purified by flash chromatography with silica gel (isocratic elution: 1:9 CH₃OH/CH₂Cl₂), to give the desired product as a flaky cream coloured solid (0.124 g, 98%). TLC R_{f} (85:15 CH₂Cl₂/CH₃OH+0.1% AcOH)=0.48; ¹H NMR (300.13 MHz, acetone-d₆): δ 60.5-2.5 (bm BH), 1.30 (m, CH₃), 3.34 (m, CH₂), 3.65 (s, NH), 5.11 (m, CH₂), 7.26(m, C₆H₅); ¹³C{¹H} NMR (50.3 MHz, acetone-d₆,): δ 7.55, 15.46, 49.73, 52.80, 65.94, 66.69, 66.98, 67.54, 127.72, 128.09, 128.32, 128.45, 128.86, 129.01, 137.76, 137.96, 156.18, 157.20; ¹¹B{¹H} NMR (96.3 MHz, acetone-d₆): δ 19.65, 18.93, −2.85, −7.07, −20.43, −23.28, −34.13, −37.72; FTIR (KBr, cm⁻¹): ν 3364, 3002, 2535, 1755, 1706; HRMS (ESI-Q-TOF): Calcd for C₁₉B₁₀H₂₆N₂O₆: 432.2780. Found: 432.27 82.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

We claim:

1. A method of preparing metal-carborane complexes comprising reacting a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, with a nidocarborane in the presence of a hard base.

2. The method according to claim 1, wherein M is selected from radioisotopes of Tc, Re, Rh, Cr, Mo, Mn, Os, Ir and Ru.

3. The method according to claim 2, wherein M is selected from radioisotopes of Tc and Re.

4. The method according to claim 1, wherein X^{m} is independently selected from Cl⁻(m=−1), Br⁻¹(m=−1), PR₃ (m=0), RCN (m=0), NOₓ^{y}(x 32 1, 2; y=1, −1) and H₂O (m=0).

5. The method according to claim 4, wherein all three X^{m} ligands are Br⁻(m=−1) or H₂O (m=0).

6. The method according to claim 1, wherein one or more of the CO ligands is substituted with a ligand that is isoelectronic and isolobal therewith.

7. The method according to claim 6, wherein the ligand that is isoelectronic and isolobal with CO is selected from one or more of NO⁺, PR₃, RNC and RCN, wherein R is an alkyl group, an aryl group or any biomolecule.

8. The method according claim 1, wherein the nido-carborane is unsubstituted or substituted with one or more linker groups attached to one or more carbon and/or boron atoms.

9. The method according to claim 8, wherein the one or more linker groups are attached to the carbon atoms in the nido-carborane.

10. The method according to claim 9, wherein one linker group is attached to one of the carbon atoms in the nido-carborane.

11. The method according to claim 8, wherein the one or more linker groups has a biological targeting molecule attached thereto.

12. The method according to claim 1, wherein the nido-carborane is selected from compounds 1a, 1b and 1c.

13. The method according to claim 1, wherein the nido-carborane is incorporated within the structure of a biological targeting ligand.

14. The method according to claim 1, wherein the hard base is selected from O²⁻, Cl⁻, F⁻, CH₃COO⁻, NO₃⁻, ClO₄⁻, SO₄²⁻, NH₃ and RNH₂, wherein R is any suitable alkyl or aryl group.

15. The method according to claim 14, wherein the hard base is a source of fluoride (F⁻).

16. The method according to claim 1, comprising generating the nido-carborane from a corresponding dicarba-nido-undecaborate by treatment with the hard base in aqueous solution.

17. The method according to claim 1, wherein an aqueous solution of a salt of the formula [M(CO)₃(X^{m})₃]^{(1+3m)} is combined with the hard base and warmed to a temperature of about 60-100° C., and this solution is added to a solution comprising the nido-carborane at a temperature of about 60-100° C., and the temperature maintained until reaction completion.

18. The method according to claim 1, wherein an aqueous solution of a salt of the formula [M(CO)₃(X^{m})₃]^{(1+3m)} is combined with the hard base and this solution is added to a solution comprising the nido-carborane and the combined solutions are heated in a microwave until reaction completion.

19. A kit for use in the preparation of the salts of formula [M(CO)₃(X^{m})₃]^{(1+3m)}, wherein M is a radioisotope of Tc or Re and X is H₂O , comprising potassium boranocarbonate (K₂H₃BCO₂), Na₂B₄O₇.10H₂O, a hard base and a suitable buffering reagent.

20. A nido-carborane of the formula 1c.

21. A metal carborane complex of the formula 2c or 2d.

22. A method of preparing metal-carborane complexes comprising reacting a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, with a closo-carborane in the presence of a hard base.

23. A method for stabilizing a salt of the formula:
[M(CO)₃(X^{m})₃]^{(1+3m)},
wherein M is selected from a radioisotope of rhenium (Re), technetium (Tc) and any other radioisotope binding in the same fashion, X is the same or different and is, independently, any suitable ligand and m is the formal charge for ligand X, comprising combining the salt with a fluoride anion.
